SEQUENCE LISTING (3 sequences)

SEQ ID NO: 1 - protein, 438 aa, Cortinarius sp.
MTSTLRTLALTILSGVDAIEAAYAQTETPVPSLLDPFVPSPLDRDNTLVDAQRVVASAAAQLIAHARQPMDSIIEQSMSMYTTALLGFVVDAHIPDILKTAGRQGLAAKDIAVSAGVDAGKLARVLRYLAARHIFTEVSPNVFANNRISSILAKTKPFDEIKSSPQVPELGVTSLIGHLADESLKSAPFISSYLLDSKGYASPFNAAIGSPSTMWEWYTEPENVHRGLRFSASMKGSAARYTAETFTSAFDWAAIKPGSVVVDVGGGAGNVTVHLAKAFSHLNYVVQDLSSVITDTAEKVWAAECPEKISNGSVTLQVHDFFTAQPVKSAAVYYMRFVLHDWDDEKCIDIMKKLRVAAGPSSKLIVWERYVPYACDTTDFKAGAVLLPLTALDMHMMSMLDGEERMASELIRLGKAAGWKCEEEKNGQWLMSVVFSPE

SEQ ID NO: 2 - protein, 454 aa, Cortinarius sp.
MASQLTALANIISSSVDVLTKACSQKGLQFPDLDDPLQPSPPLDPSLVEHQALIVAAATQISAIVRPPVQTFTELAFGIYGTSILAFVVDTNIADILKEAGPDGLHVRDIAAKNNVDFSYIARVLRFLATRHIFKELSPDIFANNRLSSLLVKAKTLEEIHADPDARFDDAPYAAFISRNGDEALPSSVFLSSFMQNPGQHAAPFNIAFKTDAKLWDWYEQPENNWRGRRFRTVMKDNASQLFIKADLIDGISAASLNKDDLIVDVGGSLGKVTHALYSEMPQYRYVVQDLETLIEGGKQFWKEVSPDALASGRVELQVHNFFTPEPVKGAGLYFLRFIMHDWPDRDAQVILKHLREAANSSSKLIICDSLAVHTCESSAITAASGNKVPYPLLPCLGVAGAGFVTDMDQQMLNLYNGQERTERDFIALGEAAGWKLESVFRKTAFPSFLYSAI

SEQ ID NO: 3 - protein, 438 aa, Cortinarius semisanguineus
MTSTLRTLALTILSGVDAIEAAYSQADIPVPSLYEPFVPSPLDRDNALVDAQRVVANAAAQLIAQARQPMDSIIEQSFSMYTTALLGFVVEAHIPDILKTVGPQGLAAKEIAVLARVDAGKLARVLRYLAARHIFTEVSPNVFANNRISSILAKTRSFEEIKSSPQVHELGVTSLVGHLADESLKSVPFISSFLLDPKGYASPFNAGIGSPATMWEWYTEPENVQRGLRFSASMKGSAARYTSETFTSAFDWAAIKDGSTVVDVGGGAGNVTVHLAKAFSHLNYVVQDLSSVITDTAEKVWAAECPEKIADGSVSLQVHDFFTEQPIKSAAVYYMRFVLHDWDDEKCIDIMRKLRVAAGPSSSLIVWERFVPYACDTTDFKAAAVLLPLTALDMHMMSMLDGEERMASELVRLGKAAGWKFEEKKVGQWLMTLVFSPE

(19) United States
(12) Patent Application Publication
(10) Pub. No.: US 2026/0347800 A1
(43) Pub. Date: Nov. 5, 2026

(54) PROCESS TO PRODUCE ANTHRAQUINONE PIGMENTS AND DYES

(71) Applicant: TEKNOLOGIAN TUTKIMUSKESKUS VTT OY, Espoo (FI)

(72) Inventors: Istvan MOLNAR, Espoo (FI); Satu HILDITCH, Espoo (FI); Dominik MOJZITA, Espoo (FI); Merja PENTTILÄ, Espoo (FI); Pradhuman JETHA, Espoo (FI); Jorg DE RUIJTER, Espoo (FI); Mervi TOIVARI, Espoo (FI)

(21) Appl. No.: 19/491,082

(22) PCT Filed: Jun. 13, 2024
(86) PCT No.: PCT/FI2024/050306

§ 371 Date: Dec. 8, 2025

(30) Foreign Application Priority Data
Jun. 15, 2023 (FI) 20235675

(51) Int. Cl. C12N 9/10; C12N 15/81; C12P 7/66; C12R 1/69; C12R 1/865; C12R 1/885

(52) U.S. Cl. CPC C12N 9/1007; C12N 15/815; C12P 7/66; C12R 2001/69; C12R 2001/865; C12R 2001/885; C12Y 201/01

(57) ABSTRACT

The present disclosure relates to O-methyltransferase enzymes. The present disclosure also relates to polypeptides having O-methyltransferase activity, recombinant vectors, host cells, and enzyme preparations thereof. The present disclosure also relates to a process for producing O-methylanthraquinones using O-methyltransferase enzymes as well as their use as pigments and dyes.

### FIELD OF THE DISCLOSURE

The present disclosure relates to O-methyltransferase enzymes. The present disclosure also relates to polypeptides having O-methyltransferase activity, recombinant vectors, host cells, and enzyme preparations thereof. The present disclosure also relates to a process for producing O-methylanthraquinones using O-methyltransferase enzymes as well as their use as pigments and dyes.

### BACKGROUND

Anthraquinone colorant extracts from the basidiomycete cap mushroom Cortinarius spp. (formerly Dermocybe spp.) have been used as traditional, artisan textile dyes. These anthraquinones yield vivid, orange to deep red colors. They are stable during storage conditions, light (UV) and pH stable and show little fading or leaching from textiles and plastics (Räisänen 2018). While early intermediates of the anthraquinone biosynthetic pathway such as endocrocin and emodin show some genotoxicity and eukaryotic cell toxicity, several other anthraquinones produced by Cortinarius spp. are nontoxic to mammalian cells and are not mutagenic. Furthermore, these intermediates and derivatives are produced by Cortinarius mushrooms in an uncontrollable manner, and thus are present in the various extracts as an uncharacterized and variable mixture of anthraquinones. The presence of many anthraquinones in the extracts in variable amounts together with other, unidentified metabolites of the mushroom also leads to inconsistent color hues and variable dyeing outcomes with the natural extracts, in addition to variable and uncontrolled safety profiles for these colorants.

The yellow anthraquinone pigments endocrocin and emodin are produced by many microorganisms and plants from the polyketide atrochrysone carboxylic acid. Spontaneous loss of water and aromatization through keto-enol tautomerization then affords endocrocin anthrone. This compound undergoes spontaneous or enzyme-catalyzed oxidation to afford endocrocin, a stable anthraquinone. Alternatively, a decarboxylase enzyme may convert atrochrysone carboxylic acid to atrochrysone, which upon spontaneous loss of water affords emodin anthrone. This is then oxidized enzymatically and/or spontaneously to yield emodin, another stable anthraquinone (Griffiths et al. 2016).

Atrochrysone carboxylic acid, emodin and endocrocin are converted to many other secondary metabolites by subsequent biochemical reactions in a variety of fungi (for example, to asperthecin, monodictyphenone, geodin, cladofulvin etc.) and plants (for example, to chrysophanol, aloe emodin, rufigallol, etc.). In the basidiomycete fungi Cortinarius spp., atrochrysone carboxylic acid is produced by a Type I iterative, multidomain nonreducing polyketide synthase (nrPKS) enzyme that has an integrated thioesterase (TE) domain at its C-terminus. After atrochrysone carboxylic acid is converted to endocrocin and emodin, these anthraquinones undergo further modifications (“tailoring events”) catalyzed by various enzymes. Thus, 8-O-methylation of endocrocin yields dermolutein, while 6-O-methylation of emodin yields physcion. These tailoring events affect the light absorption maxima (hence the color), the solubility, the surface adhesion, the bioavailability, and the bioactivities of the endocrocin and emodin derivatives. The enzymes responsible for these tailoring events have not been identified from basidiomycetes.

As opposed to chemical synthesis, enzymatic O-methylation offers regio- and stereospecific outcomes even for highly complex and reactive scaffolds without resorting to expensive multistep protection/deprotection strategies. It also proceeds with a high yield under mild, environmentally friendly reaction conditions, and does not have to contend with the formation of C-methylated side products in the case of phenolic substrates. In the biotechnology industries, O-methylation is conducted by utilizing in vivo biocatalytic platforms instead of in vitro reconstituted enzymatic reactions because the availability of the methyl donor co-substrate S-adenosyl-methionine (SAM) is limited, and its in vitro regeneration is inefficient.

Räisänen 2018 discloses extraction of anthraquinone compound mixtures from Cortinarius sanguineus and Cortinarius semisanguineus but does not disclose any specific enzymes capable of catalyzing the reactions leading to product formation. Patent publication CN 108004193 relates to a method for synthesizing emodin. Sun et al. 2019 disclose reconstituting the biosynthetic pathway of endocrocin and emodin in S. cerevisiae by combining enzymes from different sources. The pathway includes e.g. decarboxylases and polyketide synthases. Sun et al. 2019 disclose a method for performing the first steps leading to endocrocin and emodin production.

Parajuli et al. 2018 report methylation of emodin by the SpOMT7740 O-methyltransferase of Streptomyces peucetius, but the product was not isolated, and the position of the methylation was not determined. Xue et al. 2022 disclose characterization of emodin-O-methyltransferase (GedA) from Aspergillus terreus. This enzyme is heterologously expressed in E. coli. Anderson 1986 discloses converting emodin to physcion by a methyltransferase of Aspergillus parasiticus but does not characterize the enzyme. Qi et al. 2022 disclose engineering of Aspergillus terreus to produce physcion. Yao et al. 2023 disclose engineering of Aspergillus nidulans to produce physcion.

It is an object of the present disclosure to solve or alleviate at least some of the problems of the prior technology to produce O-methylanthraquinones.

### SUMMARY OF THE DISCLOSURE

The present disclosure provides novel O-methyltransferase enzymes. The disclosure also provides production of O-methylanthraquinones. Especially the disclosure provides a method of producing specific dermolutein and physcion analogues by modifying the well-known anthraquinone scaffolds of endocrocin and emodin.

In an aspect, the present disclosure relates to an O-methyltransferase enzyme, wherein said O-methyltransferase enzyme has O-methyltransferase activity on an anthraquinone and comprises an amino acid sequence having at least 60% identity, preferably at least 70% identity, more preferably at least 80% identity, most preferably at least 90% identity to an amino acid sequence as defined in any of SEQ ID NO: 1, SEQ ID NO: 2, or SEQ ID NO: 3.

In another aspect, the present disclosure relates to an isolated nucleic acid molecule comprising a polynucleotide sequence which encodes an O-methyltransferase enzyme and is selected from the group consisting of:
a) a nucleic acid molecule encoding a polypeptide having O-methyltransferase activity on an anthraquinone and comprising the amino acid sequence as defined in SEQ ID NO: 1.
b) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 1.
c) a nucleic acid molecule encoding a polypeptide having O-methyltransferase activity on an anthraquinone and comprising the amino acid sequence as defined in SEQ ID NO: 2.
d) a nucleic acid molecule encoding a polypeptide having O-methyltransferase activity and at least 60% identity, preferably at least 70% identity, more preferably at least 80% identity, most preferably at least 90% identity to the amino acid sequence as defined in SEQ ID NO: 2.
e) a nucleic acid molecule encoding a polypeptide having O-methyltransferase activity on an anthraquinone and comprising the amino acid sequence as defined in SEQ ID NO: 3.
f) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in or SEQ ID NO: 3.

In another aspect, the present disclosure relates to a recombinant expression vector, wherein the expression vector comprises said isolated nucleic acid described above operably linked to at least one regulatory sequence capable of directing expression of said O-methyltransferase in a suitable host.

In another aspect, the present disclosure relates to a host cell, wherein the host cell comprises a nucleic acid molecule according to the present disclosure, or the recombinant expression vector.

In an aspect, the present disclosure relates to an enzyme preparation comprising O-methyltransferase.

In an aspect, the present disclosure relates to a process of producing a compound which is an O-methylanthraquinone, wherein the process comprises providing an anthraquinone, providing a recombinant host cell according to the present disclosure; culturing the host cell under conditions to produce an O-methyltransferase which converts the anthraquinone to O-methylanthraquinone, wherein the host cell comprises a nucleic acid sequence encoding O-methyltransferase exhibiting activity for catalyzing an O-methylation reaction on said anthraquinone; converting enzymatically the anthraquinone to an O-methylanthraquinone, and optionally recovering said O-methylanthraquinone.

In an aspect, the present disclosure relates to O-methylanthraquinone obtainable by the process disclosed in the present disclosure.

In an aspect, the present disclosure relates to use of the O-methylanthraquinone according to the present disclosure as a colorant in textiles, plastics, pigments, dyes, food, feed, or cosmetics industries, or any related industries.

In an aspect, the present disclosure relates to a colorant composition, wherein said composition comprises an O-methylanthraquinone obtained according to the process of the present disclosure.

### BRIEF DESCRIPTION OF THE FIGURES

FIG. 1 illustrates the biosynthetic gene cluster of Cortinarius sp. KIS-3 that encodes PKS 1168005 (PKS3), identified using C. odorifer nrPKS as the bait sequence. A: Monooxygenase; B: Monooxygenase; C: Monooxygenase/Halogenase; D: O-methyltransferase; E: Oxidase; F: Transporter; G: 1168005_PKS; H: Hypothetical protein; I: Transporter; J: Transporter.

FIGS. 2A, 2B and 2C illustrate LC-MS detection of emodin and endocrocin in S. cerevisiae PKS3 (H6111) culture supernatants and cell extracts. FIG. 2A: Endocrocin in culture supernatant, FIG. 2B: Emodin in cell extract; FIG. 2C: Emodin standard.

FIG. 3 illustrates an increase in emodin production by co-expression of the Aspergillus fischeri decarboxylase in S. cerevisiae.

FIGS. 4A and 4B illustrate LC-MS detection of dermolutein in S. cerevisiae PKS3-OMT2 (H6115) culture supernatant compared to a strain expressing only PKS3 (H6111). FIG. 4A: Dermolutein in H6115 culture supernatant; FIG. 4B: Absence of dermolutein in H6111 culture supernatant.

FIGS. 5A and 5B illustrate LC-MS detection of physcion in S. cerevisiae PKS3-OMT4 (H6117) culture cell extracts, compared with that of physcion standard. FIG. 5A: Physcion in H6117 culture cell extract; FIG. 5B: Physcion standard.

FIG. 6A illustrates MS/MS fragmentation pattern for endocrocin: Endocrocin standard data available from literature Massbank of North America. Available at: https://mona.fiehnlab.ucdavis.edu/spectra/display/CCMSLIB00004751377 (Accessed: Jan. 30, 2023). MoNA ID-CCMSLIB00000848193.

FIG. 6B illustrates MS/MS fragmentation pattern for endocrocin: Endocrocin detected in fermentation extract of S. cerevisiae PKS3-OMT4 (H6117)

FIGS. 7A and 7B illustrate MS/MS fragmentation pattern comparison for emodin. FIG. 7A: Emodin detected in S. cerevisiae PKS3-OMT4 (H6117) fermentation extract; FIG. 7B: Authentic emodin standard.

FIGS. 8A and 8B illustrate MS/MS fragmentation pattern comparison for physcion.

FIG. 8A: Physcion detected in S. cerevisiae PKS3-OMT4 (H6117) fermentation extract;

FIG. 8B: authentic physcion standard.

FIGS. 9A and 9B illustrate MS/MS fragmentation pattern comparison for dermolutein.

FIG. 9A: Dermolutein detected in S. cerevisiae PKS3-OMT2 (H6115) fermentation sample; FIG. 9B: Authentic dermolutein standard.

FIG. 10 illustrates a plasmid harbouring NpgA-ACC1 bi-directional SES expression cassette having flanks to LEU loci of S. cerevisiae. Expression cassette released by digesting the plasmid with NotI.

FIG. 11 illustrates a plasmid harbouring Cortinarius sp. KIS-3 PKS 1168005 (PKS3) and Cortinarius sp. KIS-3 OMT 1191744 (OMT2) bi-directional SES expression cassette having flanks to HIS loci of S. cerevisiae. Expression cassette released by digesting the plasmid with NotI.

FIG. 12 illustrates the biosynthetic pathway to yield dermolutein and physcion, including the O-methylation reactions catalyzed by OMT2 of Cortinarius sp. KIS-3, OMT5 of C. semisanguineus and OMT4 of Cortinarius sp. KIS-3.

FIG. 13 illustrates conversion of purified endocrocin to dermolutein with recombinant S. cerevisiae. (A) Authentic dermolutein standard. (B) Dermolutein from a S. cerevisiae B14443 culture. (C) MS/MS spectrum of dermolutein standard. (D) MS/MS spectrum of dermolutein peak from S. cerevisiae B14443 culture.

FIG. 14 illustrates conversion of purified emodin to physcion with recombinant S. cerevisiae.

(A) Authentic physcion standard. (B) Physcion from a S. cerevisiae B14444 culture. (C) MS/MS spectrum of physcion standard. (D) MS/MS spectrum of physcion peak from a S. cerevisiae B14444 culture.

FIG. 15 illustrates the production of dermolutein with recombinant Aspergillus fumigatus. (A) Dermolutein standard. (B) Dermolutein from Aspergillus fumigatus B13990 culture.

FIG. 16 illustrates production of dermolutein with recombinant Aspergillus fumigatus. (A) Physcion standard. (B) Physcion from Aspergillus fumigatus B13992 culture.

FIG. 17 illustrates production of dermolutein with purified OMT2 or OMT5 enzymes. (A) Authentic dermolutein standard. (B) Dermolutein from in vitro enzymatic reaction with endocrocin as the substrate. (C) MS/MS spectrum of dermolutein standard. (D) MS/MS spectrum of dermolutein peak from an in vitro enzymatic reaction with endocrocin as the substrate.

FIG. 18 illustrates production of physcion with purified OMT4 enzyme. (A) Authentic physcion standard. (B) Physcion peak from in vitro enzymatic reaction with emodin as the substrate. (C) MS/MS spectrum of physcion standard. (D) MS/MS spectrum of physcion peak from an in vitro enzymatic reaction with endocrocin as the substrate.

FIG. 19 illustrates a sequence alignment of SEQ ID NO: 2 (OMT4), SEQ ID NO: 3 (OMT5), and GedA. Residues for the substrate binding site (1), the SAM binding site (2), and the catalytic active site (3) are highlighted.

FIG. 20A illustrates a structure alignment of structure models of SEQ ID NO: 1 and SEQ ID NO: 3 (OMT2 or OMT5; completely overlapping cartoons shown in black), generated with AlphaFold, and the published crystal structure of GedA (white cartoon, Xue et al. 2022).

FIG. 20B illustrates S-adenosylhomocysteine (SAH) was docked into the structure models of SEQ ID NO: 1 and SEQ ID NO: 3 using Autodock Vina, and residues predicted to form the binding site are show in sticks for SEQ ID NO: 3 (OMT5). Identical SAH pose and binding site residues were obtained with SEQ ID NO: 1 (OMT2). Predicted hydrogen bonds are indicated with dashed lines.

FIG. 21 illustrates predicted substrate binding sites in SEQ ID NO: 1 and SEQ ID NO: 3 (OMT2 and OMT5, completely overlapping models). The co-product S-adenosylhomocysteine (SAH), the substrate (endocrocin), the substrate binding sites and catalytic residues of SEQ ID NO: 1 and SEQ ID NO: 3 (completely overlapping and identical residues) are also shown. Predicted hydrogen bonds and key interactions are indicated with dashed lines.

FIG. 22A illustrates a structure alignment of structure models of SEQ ID NO: 2 (OMT4; shown in black), generated with AlphaFold, and the published crystal structure of GedA (white cartoon, Xue et al. 2022) of A. terreus.

FIG. 22B illustrates S-adenosylhomocysteine (SAH) was docked into the structure models of SEQ ID NO: 2 using Autodock Vina, and residues predicted to form the binding site are shown for SEQ ID NO: 2 (OMT4). Predicted hydrogen bonds are indicated with dashed lines.

FIG. 23 illustrates predicted substrate binding sites in SEQ ID NO: 2. The co-product S-adenosylhomocysteine (SAH), the substrate (emodin), the substrate binding sites and catalytic residues of SEQ ID NO: 2 are shown. Predicted hydrogen bonds and key interactions are indicated with dashed lines.

### SEQUENCE LISTING

SEQ ID NO: 1 is a protein sequence of OMT2 of Cortinarius sp. KIS-3 catalyzing O-methylation of C8 hydroxy group of endocrocin to yield dermolutein.

MTSTLRTLALTILSGVDAIEAAYAQTETPVPSLLDPFVPSPLDRDNTLVD
AQRVVASAAAQLIAHARQPMDSIIEQSMSMYTTALLGFVVDAHIPDILKT
AGRQGLAAKDIAVSAGVDAGKLARVLRYLAARHIFTEVSPNVFANNRISS
ILAKTKPFDEIKSSPQVPELGVTSLIGHLADESLKSAPFISSYLLDSKGY
ASPFNAAIGSPSTMWEWYTEPENVHRGLRFSASMKGSAARYTAETFTSAF
DWAAIKPGSVVVDVGGGAGNVTVHLAKAFSHLNYVVQDLSSVITDTAEKV
WAAECPEKISNGSVTLQVHDFFTAQPVKSAAVYYMRFVLHDWDDEKCIDI
MKKLRVAAGPSSKLIVWERYVPYACDTTDFKAGAVLLPLTALDMHMMSML
DGEERMASELIRLGKAAGWKCEEEKNGQWLMSVVFSPE

SEQ ID NO: 2 is a protein sequence of OMT4 of Cortinarius sp. KIS-3 catalyzing O-methylation of C6 hydroxy group of emodin to yield physcion.

MASQLTALANIISSSVDVLTKACSQKGLQFPDLDDPLQPSPPLDPSLVEH
QALIVAAATQISAIVRPPVQTFTELAFGIYGTSILAFVVDTNIADILKEA
GPDGLHVRDIAAKNNVDFSYIARVLRFLATRHIFKELSPDIFANNRLSSL
LVKAKTLEEIHADPDARFDDAPYAAFISRNGDEALPSSVFLSSFMQNPGQ
HAAPFNIAFKTDAKLWDWYEQPENNWRGRRFRTVMKDNASQLFIKADLID
GISAASLNKDDLIVDVGGSLGKVTHALYSEMPQYRYVVQDLETLIEGGKQ
FWKEVSPDALASGRVELQVHNFFTPEPVKGAGLYFLRFIMHDWPDRDAQV
ILKHLREAANSSSKLIICDSLAVHTCESSAITAASGNKVPYPLLPCLGVA
GAGFVTDMDQQMLNLYNGQERTERDFIALGEAAGWKLESVFRKTAFPSFL
YSAI

SEQ ID NO: 3 is a protein sequence of OMT5 of C. semisanguineus catalyzing O-methylation of C8 hydroxy group of endocrocin to yield dermolutein.

MTSTLRTLALTILSGVDAIEAAYSQADIPVPSLYEPFVPSPLDRDNALVD
AQRVVANAAAQLIAQARQPMDSIIEQSFSMYTTALLGFVVEAHIPDILKT
VGPQGLAAKEIAVLARVDAGKLARVLRYLAARHIFTEVSPNVFANNRISS
ILAKTRSFEEIKSSPQVHELGVTSLVGHLADESLKSVPFISSFLLDPKGY
ASPFNAGIGSPATMWEWYTEPENVQRGLRFSASMKGSAARYTSETFTSAF
DWAAIKDGSTVVDVGGGAGNVTVHLAKAFSHLNYVVQDLSSVITDTAEKV
WAAECPEKIADGSVSLQVHDFFTEQPIKSAAVYYMRFVLHDWDDEKCIDI
MRKLRVAAGPSSSLIVWERFVPYACDTTDFKAAAVLLPLTALDMHMMSML
DGEERMASELVRLGKAAGWKFEEKKVGQWLMTLVFSPE

### DEFINITIONS

The term “anthraquinone” refers to an aromatic polycyclic hydrocarbon wherein R₁, R₂, R₃, R₄, R₅, R₆, R₇ and R₈ are independently selected, and each R group is a substituted or unsubstituted, saturated or unsaturated, cyclic or acyclic, aliphatic or aromatic hydrocarbon group, optionally comprising one or more heteroatoms (such as O, N, S), hydroxyl group, methoxy group or hydrogen (Formula I).

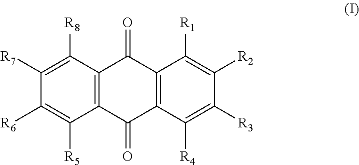

In the present disclosure anthraquinone may be selected from the group consisting of endocrocin, endocrocin analogues, emodin, and emodin analogues.

The term “endocrocin” refers to an anthraquinone where R₁, R₆ and R₅ are hydroxyl groups, R₂ is a carboxylic acid group, R₃ is a methyl group and R₄, R₅ and R₇ are hydrogen atoms (Formula II).

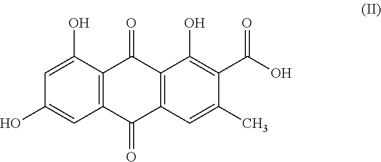 [SMILES: Cc1cc2c(c(O)c1C(=O)O)C(=O)c1c(O)cc(O)cc1C2=O]

The term “endocrocin analogue” or “endocrocin analogues” refers to compounds structurally similar to endocrocin but with substitution of one or more atoms by an atom of a different element or a functional group.

The term “emodin” refers to an anthraquinone where R₁, R₆ and R₅ are hydroxyl groups, R₃ is a methyl group and R₂, R₄, R₅ and R₇ are hydrogen atoms (Formula III).

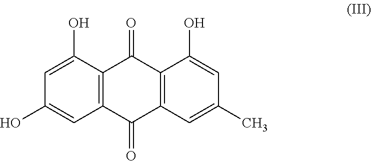 [SMILES: Cc1cc(O)c2c(c1)C(=O)c1cc(O)cc(O)c1C2=O]

The term “emodin analogue” or “emodin analogues” refers to compounds structurally similar to emodin but with substitution of one or more atoms by an atom of a different element or a functional group.

The term “atrochrysone carboxylic acid” refers to the compound with the following structure (Formula IV)

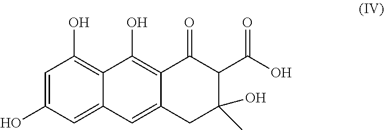 [SMILES: CC1(O)Cc2cc3cc(O)cc(O)c3c(O)c2C(=O)C1C(=O)O]

The term “an atrochrysone carboxylic acid-derived compound” refers to an anthraquinone which is obtained by modifying atrochrysone carboxylic acid.

The term “an atrochrysone carboxylic acid analogue” refers to compounds structurally similar to atrochrysone carboxylic acid but with substitution of one or more atoms by an atom of a different element or a functional group.

The term “O-methylanthraquinone” refers to an anthraquinone where an alcohol functionality of the anthraquinone is methylated to yield an anthraquinone with a methoxy group.

In the present disclosure O-methylanthraquinone may be selected from the group consisting of dermolutein, physcion, dermolutein analogues, and physcion analogues.

The term “O-methyltransferase” refers to an enzyme that catalyzes the methylation of a hydroxyl group of a substituted hydrocarbon to yield a methoxy-substituted hydrocarbon.

The term “anthraquinone O-methyltransferase activity” in the present disclosure refers to O-methyltransferase activity on an anthraquinone displayed by an O-methyltransferase. In other words, said O-methyltransferase has O-methyltransferase activity on an anthraquinone in an O-methylation reaction. Said O-methyltransferase enzyme is able to O-methylate an anthraquinone to produce an O-methylanthraquinone and comprises an amino acid sequence having at least 60% identity, preferably 70% identity, more preferably at least 80% identity, most preferably at least 90% identity to an amino acid sequence as defined in SEQ ID NO: 1, SEQ ID NO:2, or SEQ ID NO:3.

The term “dermolutein” refers to 8-O-methylendocrocin. The term “dermolutein analogue” or “dermolutein analogues” refers to compounds structurally similar to dermolutein but with substitution of one or more atoms by an atom of a different element or a functional group.

The term “physcion” refers to 6-O-methylemodin. The term “physcion analogue” or “physcion analogues” refers to compounds structurally similar to physcion but with substitution of one or more atoms by an atom of a different element or a functional group.

The term “derivative” refers to a chemical compound that can be obtained from another compound by substituting any of its constituent atoms with another atom or group of atoms using a chemical reaction.

The term “analogue” refers to a chemical compound whose structure is substantially similar to that of another chemical compound but with substitution of one or more atoms by an atom of a different element or a functional group.

The term, “host cell” means any cell type that is susceptible to transformation, transfection, transduction, mating, crossing or the like with a nucleic acid construct or expression vector comprising a polynucleotide. The term “host cell” also encompasses any progeny of a parent host cell that is not identical to the parent host cell due to mutations that occur during replication. The host cell may be a microbial or plant host cell or a non-human animal cell. The term “microbial host cell” refers to a prokaryotic or eukaryotic microorganisms such as a bacterium, a filamentous fungus or a yeast.

The term “recombinant cell” or “recombinant host cell” refers to a cell or host cell that has been genetically modified or altered to comprise a nucleic acid sequence which is not native to said cell or host cell. In an embodiment the genetic modification comprises integrating the polynucleotide in the genome of the host cell. In another embodiment the polynucleotide is exogenous to the genomic DNA in the host cell. “Recombinant host cell” designates a cell that is not found in nature, and which contains a modified genome as a result of either a deletion, insertion, or modification of one or several genetic elements.

A “recombinant nucleic acid” or “recombinant nucleic acid molecule” designates a nucleic acid (such as, e.g., DNA, cDNA, or RNA molecule) which has been engineered and is not found as such in nature. Typically, this term refers to a nucleic acid molecule comprising segments generated and/or joined together using recombinant DNA technology, such as for example molecular cloning and nucleic acid amplification. A recombinant nucleic acid molecule comprises one or more non-naturally occurring sequences, and/or contains joined nucleic acid molecules from different original sources and not naturally attached together.

The term “gene” designates any nucleic acid encoding a protein. This term encompasses DNA as well as RNA. The gene may be first prepared by e.g., recombinant, enzymatic and/or chemical techniques, and subsequently replicated in a host cell or an in vitro system. The gene typically comprises an open reading frame encoding a desired protein. The gene may contain additional sequences such as a transcription terminator, a promoter, or a signal peptide.

The term “operably linked” means a configuration in which a control sequence is placed at an appropriate position relative to a coding sequence, in such a way that the control sequence directs expression of the coding sequence.

The term “control sequence” means nucleic acid sequences necessary for expression of a gene. Control sequences may be native or heterologous. Well-known control sequences and currently used by the person skilled in the art will be preferred. Such control sequences include, but are not limited to, a leader, polyadenylation sequence, propeptide sequence, promoter, signal peptide sequence, ribosome binding site and transcription terminator. Preferably, the control sequences include a promoter and a transcription terminator.

The term “expression vector” means a DNA molecule that comprises an expression cassette. Preferably, the expression vector is a linear or circular double stranded DNA molecule.

The amino acids are herein represented by their one-letter or three-letter code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gin); R: arginine (Arg); S: serine (Ser); T: threonine (Thr); V: valine (Val); W: tryptophan (Trp) and Y: tyrosine (Tyr).

As used herein, “isolated” and “recovered” mean a substance in a form or environment that does not occur in nature. Non-limiting examples of isolated substances include (1) any non-naturally occurring substance, (2) any substance including any enzyme, variant, nucleic acid, protein, peptide or cofactor, or small molecule that is at least partially removed from one or more or all of the naturally occurring constituents with which it is associated in nature; (3) any substance modified by the hand of man relative to that substance found in nature; or (4) any substance modified by increasing or decreasing the amount of the substance relative to other components with which it is naturally associated (e.g., recombinant production in a host cell; one or multiple copies of a gene; and use of an alternative promoter to the promoter naturally associated with the gene). In an embodiment a polypeptide, enzyme, polynucleotide, host cell, a metabolite or composition of the invention is isolated.

As used herein, a “peptide” and a “polypeptide” are amino acid sequences including a plurality of consecutive polymerized amino acid residues. For the purpose of this invention, peptides are molecules including up to 20 amino acid residues, and polypeptides include more than 20 amino acid residues. The peptide or polypeptide may include modified amino acid residues, naturally occurring amino acid residues not encoded by a codon, and non-naturally occurring amino acid residues. As used herein, a “protein” may refer to a peptide or a polypeptide of any size. A protein may be an enzyme, a protein, an antibody, a membrane protein, a peptide hormone, regulator, or any other protein.

The term “polynucleotide” denotes a single- or double-stranded polymer of deoxyribonucleotide, or ribonucleotide bases read from the 5′ to the 3′ end. Polynucleotides include RNA and DNA, and may be isolated from natural sources, synthesized in vitro, or prepared from a combination of natural and synthetic molecules.

### DETAILED DESCRIPTION

The present disclosure provides novel O-methyltransferase enzymes. The present disclosure also provides methods to produce selected anthraquinones in a specific manner by utilizing fermentations with microorganisms expressing appropriate biosynthetic genes. This synthetic biological process provides a year-round, consistent, economical, and sustainable supply of defined anthraquinone pigments and dyes for the textile, plastics, paints and coatings, and food and cosmetics industries.

Especially, the present disclosure provides a novel method and a production system involving the use of specifically defined enzymes for producing dermolutein and physcion from the precursors endocrocin and emodin, respectively.

The present disclosure provides novel genes that encode enzymes that can be used to produce specific analogues of dermolutein and physcion by modifying the well-known anthraquinone scaffolds of endocrocin and emodin. The present disclosure provides novel enzymes encoded by OMT2 (SEQ ID NO:1), OMT4 (SEQ ID NO:2) and OMT5 (SEQ ID NO:3). The present disclosure provides O-methyltransferase enzymes having O-methyltransferase activity and comprising an amino acid sequence having at least 60% identity, preferably at least 70% identity, more preferably at least 80% identity, most preferably at least 90% identity to an amino acid sequence as defined in any of SEQ ID NO: 1, SEQ ID NO: 2, or SEQ ID NO: 3.

The present inventors have surprisingly found a method for recombinant production of dermolutein by fermentation of microbial strains expressing all the necessary enzymes. Enzymatic in vitro conversion including the entire biosynthetic route to end product dermolutein has not been previously disclosed or suggested. Bona fide methyltransferases to catalyze the O-methylation of endocrocin at the C8 alcohol to afford dermolutein have not been reported previously. Similarly, the O-methyltransferases of Cortinarius or other Basidiomycetes have not been identified. The inventors have also found the first O-methyltransferase from any Basidiomycetes which catalyze the O-methylation of emodin at C6 alcohol to afford physcion.

The anthraquinone colorants dermolutein and physcion and their analogues are conventionally produced by Cortinarius spp. (formerly Dermocybe spp.) mushrooms. Cortinarius anthraquinone colorants (pigments and dyes) are extracted from wild mushrooms that are seasonal and collected and processed manually. This leads to a variable and only periodic availability in amounts not sufficient for industrial manufacturing processes; relatively high prices; and uneven quality of the colorant product. Cortinarius anthraquinone products are also not pure, but complex mixtures of emodin and endocrocin derivatives and other unidentified metabolites, displaying different hues, physicochemical properties, bioactivities, and toxicity and allergenicity profiles. The pigment industry for e.g., the textiles, plastics, paints and coatings, food colorants and cosmetics market segments demand anthraquinones that are available year-round at a reasonable price in large quantities, at constant quality, and with defined chemical composition and purity. Such a supply can only be created by the synthetic biological production of anthraquinones, including those conventionally produced by Cortinarius spp., in domesticated microbial hosts, utilizing selected genes encoding appropriate biosynthetic enzymes.

The present disclosure relates to an O-methyltransferase enzyme, wherein said O-methyltransferase enzyme has O-methyltransferase activity on an anthraquinone and comprises an amino acid sequence having at least 60% identity, preferably at least 70% identity, more preferably at least 80% identity, most preferably at least 90% identity to an amino acid sequence as defined in any of SEQ ID NO: 1, SEQ ID NO:2, or SEQ ID NO: 3. In an embodiment the O-methyltransferase enzyme has O-methyltransferase activity on an anthraquinone and comprises the amino acid sequence having at least 70% identity to an amino acid sequence as defined in any of SEQ ID NO: 1, SEQ ID NO:2, or SEQ ID NO: 3. In an embodiment the O-methyltransferase enzyme has O-methyltransferase activity on an anthraquinone and comprises the amino acid sequence having at least 80% identity to an amino acid sequence as defined in any of SEQ ID NO: 1, SEQ ID NO:2, or SEQ ID NO: 3.

In an embodiment the O-methyltransferase enzyme has O-methyltransferase activity on an anthraquinone and comprises the amino acid sequence having at least 90% identity to an amino acid sequence as defined in any of SEQ ID NO: 1, SEQ ID NO:2, or SEQ ID NO:3. In an embodiment, the O-methyltransferase enzyme comprises or consists of an amino acid sequence as defined in any of SEQ ID NO:1, SEQ ID NO:2, or SEQ ID NO: 3.

In an embodiment, the O-methyltransferase enzyme has O-methyltransferase activity on an anthraquinone and comprises an amino acid sequence having at least 60% identity, preferably at least 70% identity, more preferably at least 80% identity, most preferably at least 90% identity to an amino acid sequence as defined in SEQ ID NO:1. The O-methyltransferase enzyme may comprise an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, identity to an amino acid sequence as defined in SEQ ID NO: 1.

In an embodiment the O-methyltransferase enzyme comprises an amino acid sequence having at least 60% identity to an amino acid sequence as defined in SEQ ID NO: 1. In an embodiment the O-methyltransferase enzyme comprises an amino acid sequence having at least 70% identity to an amino acid sequence as defined in SEQ ID NO: 1. In an embodiment the O-methyltransferase enzyme comprises an amino acid sequence having at least 80% identity to an amino acid sequence as defined in SEQ ID NO:1. In an embodiment the O-methyltransferase enzyme comprises an amino acid sequence having at least 90% identity to an amino acid sequence as defined in SEQ ID NO: 1.

In a preferred embodiment O-methyltransferase enzyme comprises an amino acid sequence as defined in SEQ ID NO:1. In another preferred embodiment, O-methyltransferase enzyme consists of an amino acid sequence as defined in SEQ ID NO: 1.

In an embodiment, O-methyltransferase enzyme has O-methyltransferase activity and comprises an amino acid sequence having at least 60% identity, preferably at least 70% identity, more preferably at least 80% identity, most preferably at least 90% identity to an amino acid sequence as defined in SEQ ID NO:2. The O-methyltransferase enzyme may comprise an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, identity to an amino acid sequence as defined in SEQ ID NO:2. In an embodiment the O-methyltransferase enzyme comprises an amino acid sequence having at least 60% identity to an amino acid sequence as defined in SEQ ID NO:2. In an embodiment the O-methyltransferase enzyme comprises an amino acid sequence having at least 70% identity to an amino acid sequence as defined in SEQ ID NO:2. In an embodiment the O-methyltransferase enzyme comprises an amino acid sequence having at least 80% identity to an amino acid sequence as defined in SEQ ID NO:2. In an embodiment the O-methyltransferase enzyme comprises an amino acid sequence having at least 90% identity to an amino acid sequence as defined in SEQ ID NO: 2.

In a preferred embodiment O-methyltransferase enzyme comprises an amino acid sequence as defined in SEQ ID NO:2. In another preferred embodiment, O-methyltransferase enzyme consists of an amino acid sequence as defined in SEQ ID NO: 2.

In an embodiment, O-methyltransferase enzyme has O-methyltransferase activity and comprises an amino acid sequence having at least 60% identity, preferably at least 70% identity, more preferably at least 80% identity, most preferably at least 90% identity to an amino acid sequence as defined in SEQ ID NO:3. The O-methyltransferase enzyme may comprise an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, identity to an amino acid sequence as defined in SEQ ID NO:3.

In an embodiment the O-methyltransferase enzyme comprises an amino acid sequence having at least 60% identity to an amino acid sequence as defined in SEQ ID NO:3. In an embodiment the O-methyltransferase enzyme comprises an amino acid sequence having at least 70% identity to an amino acid sequence as defined in SEQ ID NO:3. In an embodiment the O-methyltransferase enzyme comprises an amino acid sequence having at least 80% identity to an amino acid sequence as defined in SEQ ID NO:3. In an embodiment the O-methyltransferase enzyme comprises an amino acid sequence having at least 90% identity to an amino acid sequence as defined in SEQ ID NO: 3.

In an embodiment the anthraquinone is selected from the group consisting of endocrocin and endocrocin analogues. In another embodiment the anthraquinone is selected from the group consisting of emodin, and emodin analogues.

In a preferred embodiment O-methyltransferase enzyme comprises an amino acid sequence as defined in SEQ ID NO:3. In another preferred embodiment, O-methyltransferase enzyme consists of an amino acid sequence as defined in SEQ ID NO: 3.

In a preferred embodiment the O-methyltransferase enzyme is the OMT2 as defined in SEQ ID NO: 1. SEQ ID NO: 1 refers to OMT2 of Cortinarius sp. KIS-3 catalyzing O-methylation of the C8 hydroxy group of endocrocin to yield dermolutein.

In a preferred embodiment the O-methyltransferase enzyme is the OMT4 as defined in SEQ ID NO: 2. SEQ ID NO: 2 refers to OMT4 of Cortinarius sp. KIS-3 catalyzing O-methylation of the C6 hydroxy group of emodin to yield physcion.

In a preferred embodiment the O-methyltransferase enzyme is the OMT5 as defined in SEQ ID NO: 3. SEQ ID NO: 3 refers to OMT5 of C. semisanguineus catalyzing O-methylation of the C8 hydroxy group of endocrocin to yield dermolutein.

In an embodiment O-methyltransferase exhibits activity of catalyzing an O-methylation reaction of an anthraquinone at the C8 alcohol of the scaffold to yield an 8-O-methyl anthraquinone, for example, the methylation of endocrocin to afford dermolutein.

In another embodiment, O-methyltransferase exhibits activity of catalyzing an O-methylation reaction of an anthraquinone at the C6 alcohol of the scaffold to yield an 6-O-methyl anthraquinone, for example, the methylation of emodin to afford physcion.

In an embodiment, O-methyltransferase exhibits activity of catalyzing an O-methylation reaction of an endocrocin analogue to yield a dermolutein analogue.

In an embodiment, O-methyltransferase exhibits activity of catalyzing an O-methylation reaction of an emodin analogue to yield a physcion analogue.

The present disclosure relates to an isolated nucleic acid molecule comprising a polynucleotide sequence which encodes an O-methyltransferase enzyme and is selected from the group consisting of:
a) a nucleic acid molecule encoding a polypeptide having O-methyltransferase activity on an anthraquinone and comprising the amino acid sequence as defined in SEQ ID NO: 1.
b) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 1.
c) a nucleic acid molecule encoding a polypeptide having O-methyltransferase activity on an anthraquinone and comprising the amino acid sequence as defined in SEQ ID NO: 2.
d) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 2.
e) a nucleic acid molecule encoding a polypeptide having O-methyltransferase activity on an anthraquinone and comprising the amino acid sequence as defined in SEQ ID NO: 3.
f) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in or SEQ ID NO: 3.

In an embodiment the present disclosure relates to an isolated nucleic acid molecule, wherein the nucleic acid molecule comprises a polynucleotide sequence which encodes an O-methyltransferase enzyme having O-methyltransferase activity and comprising the amino acid sequence as defined in SEQ ID NO: 1, SEQ ID NO: 2, or SEQ ID NO: 3.

The present disclosure also relates to a recombinant expression vector, wherein the expression vector comprises said isolated nucleic acid molecule operably linked to at least one regulatory sequence capable of directing expression of said O-methyltransferase enzyme in a suitable host.

In an embodiment the present disclosure relates to an expression vector comprising an isolated nucleic acid molecule comprising a polynucleotide sequence which encodes an O-methyltransferase enzyme having O-methyltransferase activity and comprising the amino acid sequence as defined in SEQ ID NO:1, SEQ ID NO:2, or SEQ ID NO:3 operably linked to regulatory sequences capable of directing expression of said O-methyltransferase enzyme in a suitable host.

The present disclosure also relates to a host cell, wherein the host cell comprises a nucleic acid molecule according to the present disclosure, or the recombinant expression vector according to the present disclosure. The host cell may be a recombinant host cell.

In an embodiment, the host cell comprises a nucleic acid sequence encoding O-methyltransferase enzyme exhibiting activity for catalysing an O-methylation reaction on an anthraquinone to yield an O-methylanthraquinone.

In an embodiment, the host cell comprises a nucleic acid molecule encoding a polypeptide having O-methyltransferase enzyme activity and comprising the amino acid sequence having at least 60% identity to the amino acid sequence as defined in SEQ ID NO: 1, SEQ ID NO:2, or SEQ ID NO:3.

The host cell may comprise at least one regulatory sequence which is a promoter.

In an embodiment the host cell is selected from a group of cells that can produce an anthraquinone.

In a preferred embodiment the host cell is selected from a group of cells that can produce endocrocin or an endocrocin analogue.

In an embodiment, the host cell is selected from the group consisting of a bacterial cell, a yeast cell, a filamentous fungus cell, a plant cell, and a non-human animal cell. In a preferred embodiment, the host cell is a filamentous fungus cell or a yeast cell.

The host may be selected from the group consisting of a genus Saccharomyces, Aspergillus, Trichoderma, and Yarrowia. The host cell may be such as Saccharomyces cerevisiae, Aspergillus oryzae, Trichoderma reesei, or Yarrowia lipolytica.

The present disclosure relates to use of the recombinant host cell according to the present disclosure to produce an O-methylanthraquinone.

The present disclosure relates to a process of producing a polypeptide having O-methyltransferase activity, wherein said process comprises the steps of culturing the host cell and optionally recovering the polypeptide.

The present disclosure relates to a polypeptide having anthraquinone O-methyltransferase enzyme activity, wherein the polypeptide is encoded by the presently claimed nucleic acid sequence and which polypeptide is obtainable by the presently claimed process comprising the steps of culturing the host cell and optionally recovering the polypeptide.

The present disclosure relates to a process of obtaining an enzyme preparation, wherein the process comprises the steps of culturing a host cell and optionally recovering the polypeptide from the cells or optionally separating the cells from a culture medium.

The present disclosure relates to an enzyme preparation obtainable by the present process. In an embodiment the enzyme preparation comprises O-methyltransferase.

The present disclosure relates to an enzyme preparation, wherein the enzyme preparation comprises O-methyltransferase enzyme having anthraquinone O-methyltransferase activity and comprises an amino acid sequence having at least 60% identity, preferably at least 70% identity, more preferably at least 80% identity, most preferably at least 90% identity to an amino acid sequence as defined in any of SEQ ID NO: 1, SEQ ID NO:2, or SEQ ID NO: 3.

In an embodiment, the O-methyltransferase enzyme comprises or consists of an amino acid sequence as defined in any of SEQ ID NO: 1, SEQ ID NO:2, or SEQ ID NO:3.

The present disclosure relates to a process of producing a compound which is an O-methylanthraquinone, wherein the process comprises the steps of providing an anthraquinone; providing a recombinant host cell according to the present disclosure; culturing the host cell under conditions to produce an O-methyltransferase which converts the anthraquinone to an O-methylanthraquinone, wherein the host cell comprises a nucleic acid sequence encoding O-methyltransferase exhibiting activity for catalyzing an O-methylation reaction on said anthraquinone; converting enzymatically the anthraquinone to the O-methylanthraquinone; and optionally recovering said O-methylanthraquinone.

In an embodiment the anthraquinone is selected from the group consisting of endocrocin, emodin, endocrocin analogues, and emodin analogues.

In a preferred embodiment the anthraquinone is selected from the group consisting of endocrocin, emodin, endocrocin analogues, and emodin analogues.

In a preferred embodiment the anthraquinone is selected from the group consisting of endocrocin and endocrocin analogues.

In an embodiment the O-methylanthraquinone is selected from the group consisting of dermolutein, physcion, dermolutein analogues, and physcion analogues.

In a preferred embodiment the O-methylanthraquinone is selected from the group consisting of dermolutein and dermolutein analogues.

In a preferred embodiment the O-methylanthraquinone is selected from the group consisting of physcion and physcion analogues.

In an embodiment the host cell is selected from the group consisting of a genus Saccharomyces, Aspergillus, Trichoderma, and Yarrowia. In a preferred embodiment the host cell is selected from the group consisting of Saccharomyces cerevisiae, Aspergillus oryzae, Trichoderma reesei, or Yarrowia lipolytica.

In an embodiment the process comprises the steps of providing endocrocin; providing a recombinant host cell according to the present disclosure; culturing the host cell under conditions to produce an O-methyltransferase which converts the endocrocin to dermolutein, wherein the host cell comprises a nucleic acid sequence encoding O-methyltransferase exhibiting activity for catalyzing an O-methylation reaction on said endocrocin; converting enzymatically the endocrocin to the dermolutein; and optionally recovering dermolutein.

In an embodiment the process comprises the steps of providing emodin; providing a recombinant host cell according to the present disclosure; culturing the host cell under conditions to produce an O-methyltransferase which converts the emodin to physcion, wherein the host cell comprises a nucleic acid sequence encoding O-methyltransferase exhibiting activity for catalyzing an O-methylation reaction on said emodin; converting enzymatically the emodin to the physcion; and optionally recovering physcion.

In an embodiment, the present disclosure also relates to a process of producing a compound which is an O-methylanthraquinone, wherein the process comprises the steps of providing an anthraquinone; providing a recombinant host cell according to the present disclosure; culturing the host cell under conditions suitable to produce an O-methylanthraquinone, converting enzymatically the anthraquinone to an O-methylanthraquinone; and optionally recovering said O-methylanthraquinone.

The anthraquinone may be selected from the group consisting of endocrocin, emodin, endocrocin analogues, and emodin analogues.

In an embodiment the host cell comprises a nucleic acid sequence encoding O-methyltransferase enzyme exhibiting activity for catalyzing an O-methylation reaction on said anthraquinone.

The O-methylanthraquinone may be selected from the group consisting of dermolutein, physcion, dermolutein analogues, and physcion analogues.

The process may further comprise isolating or purifying said O-methylanthraquinone. O-methylanthraquinones may be isolated or purified using any method known by the skilled person in the art such as liquid-liquid extraction, crystallization, precipitation, freeze-drying, drying, chromatography techniques.

Conditions suitable to produce O-methylanthraquinones may be determined by the skilled person in the art according to the recombinant host cell used. The skilled person may choose suitable culture medium and growth conditions according to the host cell.

In an embodiment the process comprises the steps of providing endocrocin or an endocrocin analogue; providing a recombinant host cell according to the present disclosure; culturing the recombinant host cell comprising a nucleic acid sequence encoding O-methyltransferase enzyme exhibiting activity for catalyzing an O-methylation reaction on said endocrocin or an endocrocin analogue under conditions suitable to produce dermolutein or a dermolutein analogue; converting enzymatically endocrocin or an endocrocin analogue to dermolutein or a dermolutein analogue; and optionally recovering said dermolutein or a dermolutein analogue.

In an embodiment the recombinant host cell comprises a nucleic acid sequence encoding the O-methyltransferase enzyme comprising an amino acid sequence having at least 60% identity, preferably at least 70% identity, more preferably at least 80% identity, most preferably at least 90% identity to an amino acid sequence as defined in SEQ ID NO: 1 or SEQ ID NO: 3.

In an embodiment the process comprises the steps of providing endocrocin or an endocrocin analogue; providing a recombinant host cell selected from the group consisting of a bacterial cell, a yeast cell, a filamentous fungus cell, a plant cell, and a non-human animal cell, preferably the host cell is a filamentous fungus cell or a yeast cell; culturing the recombinant host cell comprising a nucleic acid sequence encoding O-methyltransferase enzyme exhibiting activity for catalyzing an O-methylation reaction on said endocrocin or an endocrocin analogue under conditions suitable to produce dermolutein or a dermolutein analogue, wherein the nucleic acid sequence encoding the O-methyltransferase enzyme comprises an amino acid sequence having at least 60% identity, preferably at least 70% identity, more preferably at least 80% identity, most preferably at least 90% identity to an amino acid sequence as defined in SEQ ID NO:1 or SEQ ID NO:3; converting enzymatically endocrocin or an endocrocin analogue to dermolutein or a dermolutein analogue; and optionally recovering said dermolutein or a dermolutein analogue.

In an embodiment the process comprises the steps of providing endocrocin or an endocrocin analogue; providing a recombinant host cell selected from the group consisting of a genus Saccharomyces, Aspergillus, Trichoderma, and Yarrowia; culturing the recombinant host cell comprising a nucleic acid sequence encoding an O-methyltransferase enzyme comprising an amino acid sequence having at least 60% identity, preferably at least 70% identity, more preferably at least 80% identity, most preferably at least 90% identity to an amino acid sequence as defined in SEQ ID NO: 1 or SEQ ID NO: 3; converting enzymatically endocrocin or an endocrocin analogue to dermolutein or a dermolutein analogue, and optionally recovering said dermolutein or a dermolutein analogue.

In an embodiment the process comprises the steps of providing emodin or an emodin analogue; providing a recombinant host cell according to the present disclosure; culturing the recombinant host cell comprising a nucleic acid sequence encoding O-methyltransferase exhibiting activity for catalyzing an O-methylation reaction on said emodin or an emodin analogue under conditions suitable to produce physcion or a physcion analogue; converting enzymatically emodin or an emodin analogue to physcion or a physcion analogue; and optionally recovering said physcion or a physcion analogue.

The present disclosure relates to O-methylanthraquinones obtainable by the presently disclosed process.

In a preferred embodiment the recombinant host cell comprises a nucleic acid sequence encoding the O-methyltransferase comprising an amino acid sequence having at least 60% identity, preferably at least 70% identity, more preferably at least 80% identity, most preferably at least 90% identity to an amino acid sequence as defined in SEQ ID NO:2.

In an embodiment the process comprises the steps of providing emodin or an emodin analogue; providing a recombinant host cell selected from the group consisting of a bacterial cell, a yeast cell, a filamentous fungus cell, a plant cell, and a non-human animal cell, preferably the host cell is a filamentous fungus cell or a yeast cell; culturing the recombinant host cell comprising a nucleic acid sequence encoding O-methyltransferase exhibiting activity for catalyzing an O-methylation reaction on said emodin or an emodin analogue under conditions suitable to produce physcion or a physcion analogue, wherein the nucleic acid sequence encoding the O-methyltransferase enzyme comprises an amino acid sequence having at least 60% identity, preferably at least 70% identity, more preferably at least 80% identity, most preferably at least 90% identity to an amino acid sequence as defined in SEQ ID NO:2; converting enzymatically emodin or an emodin analogue to physcion or a physcion analogue; and optionally recovering said physcion or a physcion analogue.

In an embodiment the process comprises the steps of providing emodin or an emodin analogue; providing a recombinant host cell selected from the group consisting of a genus Saccharomyces, Aspergillus, Trichoderma, and Yarrowia; culturing the recombinant host cell comprising a nucleic acid sequence encoding an O-methyltransferase enzyme comprising an amino acid sequence having at least 60% identity, preferably at least 70% identity, more preferably at least 80% identity, most preferably at least 90% identity to an amino acid sequence as defined in SEQ ID NO:2; converting enzymatically emodin or an emodin analogue to physcion or a physcion analogue; and optionally recovering said physcion or a physcion analogue.

The present disclosure also relates to a total biosynthesis of O-methylanthraquinones, i.e., production of emodin or endocrocin in situ by the same cell that also expresses the O-methyltransferase enzyme. The production of emodin and emodin analogues, or endocrocin and endocrocin analogues may be from genes/enzymes natively present in the cell, or the production of emodin and emodin analogues, or endocrocin and endocrocin analogues may be from genes inserted to the cell.

The present disclosure relates to a process of producing O-methylanthraquinone, wherein the process comprises the steps of producing emodin or an emodin analogue in a recombinant host cell; culturing the recombinant host cell comprising a nucleic acid sequence encoding O-methyltransferase exhibiting activity for catalyzing an O-methylation reaction on said emodin or emodin analogue under conditions suitable to produce physcion or a physcion analogue; converting enzymatically emodin or an emodin analogue to a O-methylanthraquinone; and optionally recovering said O-methylanthraquinone.

In an embodiment the process comprises the steps of producing emodin or an emodin analogue in a recombinant host cell; culturing the recombinant host cell comprising a nucleic acid sequence encoding O-methyltransferase exhibiting activity for catalyzing an O-methylation reaction on said emodin or emodin analogue under conditions suitable to produce physcion or a physcion analogue; converting enzymatically emodin or an emodin analogue to physcion or a physcion analogue; optionally recovering said physcion or a physcion analogue.

In a preferred embodiment the recombinant host cell comprises a nucleic acid sequence encoding the O-methyltransferase comprising an amino acid sequence having at least 60% identity, preferably at least 70% identity, more preferably at least 80% identity, most preferably at least 90% identity to an amino acid sequence as defined in SEQ ID NO:2.

In an embodiment the process comprises the steps of producing emodin or an emodin analogue in a recombinant host cell selected from the group consisting of a genus Saccharomyces, Aspergillus, Trichoderma, and Yarrowia; culturing the recombinant host cell comprising a nucleic acid sequence encoding O-methyltransferase exhibiting activity for catalyzing an O-methylation reaction on said emodin or emodin analogue under conditions suitable to produce physcion or a physcion analogue, wherein the O-methyltransferase comprises an amino acid sequence having at least 60% identity, preferably at least 70% identity, more preferably at least 80% identity, most preferably at least 90% identity to an amino acid sequence as defined in SEQ ID NO:2; converting enzymatically emodin or an emodin analogue to physcion or a physcion analogue; optionally recovering said physcion or a physcion analogue.

The present disclosure relates to a process of producing O-methylanthraquinone, wherein the process comprises the steps of producing endocrocin or an endocrocin analogue in a recombinant host cell; culturing the recombinant host cell comprising a nucleic acid sequence encoding O-methyltransferase exhibiting activity for catalyzing an O-methylation reaction on said endocrocin or an endocrocin analogue under conditions suitable to produce dermolutein or a dermolutein analogue; converting enzymatically endocrocin or an endocrocin analogue to a O-methylanthraquinone; and optionally recovering said O-methylanthraquinone.

In another embodiment the process comprises the steps of producing endocrocin or an endocrocin analogue in a recombinant host cell; culturing the recombinant host cell comprising a nucleic acid sequence encoding O-methyltransferase exhibiting activity for catalyzing an O-methylation reaction on said endocrocin or an endocrocin analogue under conditions suitable to produce dermolutein or a dermolutein analogue; converting enzymatically endocrocin or an endocrocin analogue to dermolutein or a dermolutein analogue; optionally recovering said dermolutein or a dermolutein analogue.

In an embodiment the recombinant host cell comprises a nucleic acid sequence encoding the O-methyltransferase enzyme comprising an amino acid sequence having at least 60% identity, preferably at least 70% identity, more preferably at least 80% identity, most preferably at least 90% identity to an amino acid sequence as defined in SEQ ID NO:1 or SEQ ID NO: 3.

In another embodiment the process comprises the steps of producing endocrocin or an endocrocin analogue in a recombinant host cell selected from the group consisting of a genus Saccharomyces, Aspergillus, Trichoderma, and Yarrowia; culturing the recombinant host cell comprising a nucleic acid sequence encoding O-methyltransferase exhibiting activity for catalyzing an O-methylation reaction on said endocrocin or an endocrocin analogue under conditions suitable to produce dermolutein or a dermolutein analogue, wherein the O-methyltransferase enzyme comprises an amino acid sequence having at least 60% identity, preferably at least 70% identity, more preferably at least 80% identity, most preferably at least 90% identity to an amino acid sequence as defined in SEQ ID NO:1 or SEQ ID NO:3; converting enzymatically endocrocin or an endocrocin analogue to dermolutein or a dermolutein analogue; and optionally recovering said dermolutein or a dermolutein analogue.

The present disclosure relates to an O-methylanthraquinone obtainable by the disclosed process. In an embodiment the disclosure relates to dermolutein, a dermolutein analogue, physcion, or a physcion analogue obtainable by the disclosed process.

The present disclosure relates to a colorant composition, wherein said composition comprises the presently disclosed O-methylanthraquinone. The colorant may further comprise further ingredients or additives, which are conventionally used in such colorants and which a skilled person in the art can choose.

The present disclosure relates to use of the O-methylanthraquinone as a colorant in e.g., the textiles, plastics, pigments, dyes, food, feed, and cosmetics industries as well as in other suitable industries.

The present disclosure relates to an O-methylanthraquinone production system, wherein the system comprises a production unit containing the host cell, and a control unit comprising controlling means for operating the production unit.

By the term “identity” is here meant the identity between two amino acid sequences compared to each other within the corresponding sequence region having approximately the same number of amino acids. For example, the identity of a mature sequence of the two amino acid sequences may be compared. The amino acid sequences of the two molecules to be compared may differ in one or more positions, which however does not alter the biological function or the gross structure of the molecules. Such variation may occur naturally in different organisms or due to mutations in the amino acid sequence or they may be achieved by specific mutagenesis. The variation may result from deletion, substitution or insertion of one or more positions in the amino acid sequence. The identity of the sequences is measured by using BLASTP alignment with default settings as defined at the National Center for Biotechnology Information Basic Local Alignment Search Tool (BLAST, https://blast.ncbi.nlm.nih.gov/Blast.cgi) (Protein Weight Matrix: BLOSUM62, Gap Existence: 11, Gap extension: 1, using conditional compositional score matrix adjustment).

The present disclosure provides novel recombinant microbial strains that express novel O-methyltransferase enzymes to produce dermolutein, physcion, dermolutein analogues or physcion analogues upon fermentation. Said O-methyltransferase enzymes have O-methyltransferase activity and comprise an amino acid sequence having at least 60% identity, preferably at least 70% identity, more preferably at least 80% identity, most preferably at least 90% identity to an amino acid sequence as defined in any of SEQ ID NO: 1, SEQ ID NO:2, or SEQ ID NO:3.

The present disclosure provides novel recombinant microbial strains that express said O-methyltransferase enzymes to produce dermolutein, physcion, dermolutein analogues or physcion analogues by biocatalysis upon feeding endocrocin, emodin or their analogues to said recombinant strains upon fermentation.

The present disclosure provides novel recombinant microbial strains that express said enzymes to produce dermolutein, physcion, dermolutein analogues or physcion analogues by in vitro enzymatic reactions, upon isolating said enzymes from said strains and setting up in vitro reactions with appropriate substrates, co-substrates and/or cofactors and said enzymes.

The present disclosure provides purified dermolutein, physcion, dermolutein analogues or physcion analogues as pigments with defined chemical composition and purity, and reduced toxicity compared to endocrocin and emodin, to be used as colorants in the e.g., textile, plastics, pigments and dyes, food and feed, and cosmetics industries or in any other applications known to those skilled in the art. In other words, the compounds obtained by the presently disclosed processes may be used as pigments and colorants in industries such as textile, plastics, pigments and dyes, food and feed, and cosmetics industries and other related industries. O-methylanthraquinone, dermolutein, dermolutein analogue, physcion, or physcion analogue can be used as pigment for example in textiles, plastics, food and feed, and cosmetics.

The present disclosure provides a method to produce dermolutein, physcion, dermolutein analogues or physcion analogues as opposed to a mixture of these pigments with other, unrelated metabolites that are co-extracted from the wild Cortinarius mushrooms. The extracts from the wild mushroom may contain metabolites that are toxic or allergenic, or reduce the performance of the dermolutein, physcion, dermolutein analogues or physcion analogues as colorants in their applications.

The present disclosure provides a method to produce specific dermolutein, physcion, dermolutein analogues or physcion analogues of known purity, as opposed to an anthraquinone mixture of unknown composition from a wild mushroom. This provides specific color hues and defined physicochemical characteristics.

The present disclosure provides a method to produce dermolutein, physcion, specific dermolutein analogues or physcion analogues with better safety profiles. This provides dermolutein, physcion, dermolutein analogues or physcion analogues that have been proven to show much reduced toxicity as compared to endocrocin and emodin, both of which are constituents of colorant preparations from wild mushrooms and known to show significant toxicity.

The present disclosure provides a method to produce dermolutein, physcion, dermolutein analogues or physcion analogues all year round, not influenced by the weather or other environmental conditions, as opposed to collecting seasonal wild mushrooms.

The present disclosure provides a method to produce dermolutein, physcion, dermolutein analogues or physcion analogues in a manner that is sustainable, as opposed to exploiting nature by foraging large amounts of Cortinarius mushrooms.

In an embodiment, the production system of the present disclosure is configured to carry out the method of the present disclosure. Thus, the production system can be advantageously used to produce metabolites, including O-methylanthraquinones.

In an embodiment, the production unit is a fermenter. Preferably, the present recombinant host cell is provided inside the reactor tank of the fermenter. In an embodiment, the production unit comprises at least one fluid inlet and at least one fluid outlet, each fluid inlet and fluid outlet being in fluid connection with at least one vessel.

In an embodiment, the production unit comprises temperature controlling means for lowering and raising temperature of the production unit. In an embodiment, the control unit is configured to monitor and control cultivation of the recombinant host cells such that constitutive production of recombinant enzymes is achieved. In an embodiment, the control unit is configured to control operation of the temperature controlling means and the at least one fluid inlet and at least one fluid outlet.

In an embodiment, the present disclosure provides heterologous biosynthetic production systems for producing O-methylanthraquinones, such as dermolutein, physcion, dermolutein analogues or physcion analogues that may currently be obtained from wild Cortinarius spp. mushroom biomass.

In an embodiment, genes necessary for endocrocin and emodin production in heterologous hosts are identified from Cortinarius strains. Endocrocin and emodin can also be made by expressing appropriate Ascomycete PKSs or plant PKSs in appropriate hosts. Other host organisms, such as fungal, bacterial, plant or animal cells can also be used as cell factories. A skilled person in the art is able to choose a suitable host.

In an embodiment, the production of emodin (instead of endocrocin) is improved by using a gene from an Ascomycete encoding a decarboxylase enzyme. Other decarboxylases from other sources may also be used for such purpose. A skilled person in the art can choose a suitable enzyme.

It is apparent to a person skilled in the art that as technology advanced, the basic idea of the invention can be implemented in various ways. The invention and its embodiments are therefore not restricted to the above examples, but they may vary within the scope of the claims.

### EXAMPLES

### Example 1

Construction of Saccharomyces cerevisiae Plasmids

All the S. cerevisiae plasmids (Table 1) were constructed with Gibson assembly (NEB Gibson Assembly® Master Mix kit).

The phosphopantetheinyl transferase npgA gene of Aspergillus nidulans (GenBank: XP_663744), codon optimized for Yarrowia lipolytica was obtained from the B12434 plasmid (Table 1) by PCR using primers with compatible overhangs to the vector. The acc1** gene was procured by digesting pSK293 plasmid (Table 1) with NotI, SacI, and PvuI. These two genes, along with the SES bi-directional promoter and the B13472 (Table 1) vector fragment (including flanks to the S. cerevisiae LEU locus) were assembled using Gibson assembly to construct the NpgA-ACC1** bi-directional SES plasmid (B13474, Table 1).

The genes PKS3 (Cortinarius sp. KIS-3, JGI MycoCosm: 1168005), OMT2 (Cortinarius sp. KIS-3, JGI MycoCosm: 1191744), OMT4 (Cortinarius sp. KIS-3, JGI MycoCosm: 1127603), and OMT5 (Cortinarius semisanguineus, contig DN1090) were codon optimized for Aspergillus niger and obtained as synthetic gene blocks (Integrated DNA Technologies, USA). Similar to the NpgA-ACC1** plasmid cloning, bi-directional SES expression plasmids were constructed harbouring PKS3 and any one of the OMT genes, having the integration cassette targeted to the S. cerevisiae HIS locus.

The endocrocin decarboxylase gene of Aspergillus fischeri (GenBank: XP_001266602), codon optimized for Yarrowia lipolytica was obtained from pUC57-DC plasmid (Table 1) by PCR using primers with compatible overhangs to pLL287 vector (Table 1) having target flanks to XII-4 loci.

### Example 2

Construction of Yarrowia lipolytica Plasmids

For the construction of Y. lipolytica plasmids (YI-pPKS3-OMT5), the PKS3-OMT5 expression cassettes were procured by digesting plasmid B13682 (Table 1) with BamHI and Pm/I. The B11157 plasmid (Table 1; flanks to the Ant locus) was harvested from dam methylation negative E. coli strain JM110 and was then digested with BstXI followed by 20 minutes incubation step with Klenow polymerase to fill the sticky ends. This digested fragment was then purified using a DNA purification kit (NEB, England) and redigested with BclI (dam methylation sensitive) enzyme. The B11157-BstXI-Klenow-BclI digested fragment was then purified from the gel and ligated with the expression cassettes to construct corresponding plasmid B13696 (Table 1).

After the Gibson Assembly or ligation, plasmids were transformed to TOP10 E. coli electrocompetent cells (200-800′ Ω, 25 uF, 2.5 kV) and cells were plated on LB+Ampicillin (100 ug/mL) agar plates and incubated overnight at 37 °C. Colonies were selected and grown on 3 mL LB+Ampicillin (100 ug/mL) overnight. Plasmids were isolated using the Thermo Scientific™ GeneJET Plasmid Miniprep Kit and verified by restriction digestion and sequencing.

### Example 3

Construction of Aspergillus oryzae Plasmids

All the Aspergillus oryzae plasmids (Ao-pPKS3-OMT4/5) were constructed by classical ligation. S. cerevisiae constructs (B13482, B13483; Table 1) were digested with XhoI and OliI and the PKS3-OMT4 or the PKS3-OMT5 expression cassettes were extracted from the agarose gel using GeneJET Gel Extraction Kit. Similarly, B13677 (Table 1; flanks to amyA locus) was digested with the same restriction enzymes and the vector backbone was then procured from the gel. Both the digested fragments were then ligated using T4 DNA ligase kit (NEB, England).

### Example 4

Construction of Trichoderma reesei Plasmids

All the Trichoderma reesei plasmids (Tr-pPKS3-OMT4/5) were constructed by classical ligation. The expression cassettes were obtained by digesting A. oryzae constructs (B13681, B13682; Table 1) with AscI, PmlI, and MssI. The vector B8326 (flanks to CBH2 locus) was digested with AscI and EcoRV. Both the digested fragments were then extracted from the agarose gel and ligated as mentioned previously.

### Example 5

TABLE 1
Plasmids used in this study.

| Number | Name | Description | Reference |
|---|---|---|---|
| pSK293 | pSK293 | S. cerevisiae acc1^{S659A, S1157A} | VTT |
| | | under PDC1 promoter | depository |
| pLL164 | pLL164 | Cas9-gRNA plasmid for targeting | VTT |
| | | XII-4 loci of S. cerevisiae | depository |
| pLL287 | pLL287 | Plasmid having flanks targeted to | VTT |
| | | XII-4 loci of S. cerevisiae | depository |
| pUC57- | pUC57-DC | A. fischeri decarboxylase (Y. | VTT |
| DC | | lipolytica codon optimized) under | depository |
| | | FBA promoter | |
| B8326 | pRSET-CBH2-8BS(BM3R1)- | Vector plasmid having flanks to | VTT |
| | 201cp-CalB (AAss) | CBH2 loci of T. reesei | depository |
| B11157 | L-SES_(Yl-565 - | Vector plasmid harbouring | VTT |
| | 242cp)_(BnTAFM)_mCherry | synthetic transcription factor | depository |
| | | expression cassette and having | |
| | | flanks to ANT1 loci of Y. lipolytica | |
| B12434 | pRS426 + NpgA + TE | A. nidulans npgA (Yarrowia | VTT |
| | | lipolytica optimized) under TEF | depository |
| | | promoter | |
| B13472 | pLEU intermediate | Intermediate plasmid for pNpg- | This work |
| | | Acc1 construction (flanks to S. | |
| | | cerevisiae LEU locus) | |
| B13473 | pHIS intermediate | Intermediate plasmid for PKS3- | This work |
| | | OMT(n) construction (flanks to S. | |
| | | cerevisiae HIS locus) | |
| B13474 | pNpg-Acc1 | A. nidulans npgA and S. cerivisiae | This work |
| | | acc1^{S659A, S1157A} under SES | |
| | | promoters (cassette flanks to S. | |
| | | cerevisiae LEU locus) | |
| B13477 | pPKS3 | Cortinarius sp. KIS-3 PKS | This work |
| | | 1168005 (PKS3) under SES | |
| | | promoter (flanks to S. cerevisiae | |
| | | HIS locus) | |
| B13480 | pPKS3-OMT2 | Cortinarius sp. KIS-3 PKS | This work |
| | | 1168005 (PKS3) and Cortinarius | |
| | | sp. KIS-3 OMT 1191744 (OMT2) | |
| | | under SES promoters (flanks to S. | |
| | | cerevisiae HIS locus) | |
| B13482 | pPKS3-OMT4 | Cortinarius sp. KIS-3 PKS | This work |
| | | 1168005 (PKS3) and Cortinarius | |
| | | sp. KIS-3 OMT 1127603 (OMT4) | |
| | | under SES promoters (flanks to S. | |
| | | cerevisiae HIS locus) | |
| B13483 | pPKS3-OMT5 | Cortinarius sp. KIS-3 PKS | This work |
| | | 1168005 (PKS3) and C. | |
| | | semisanguineus OMT | |
| | | VTT_DN1090_OMT (OMT5) under | |
| | | SES promoters (flanks to S. | |
| | | cerevisiae HIS locus) | |
| B13485 | pDC | A. fischeri decarboxylase (Y. | This work |
| | | lipolytica codon optimized) under | |
| | | PDC1 promoter (flanks to S. | |
| | | cerevisiae XII-4 locus) | |
| B13677 | Ao_pAmy intermediate | Intermediate plasmid for PKS3- | This work |
| | | OMT(n) construction (A. oryzae | |
| | | constructs) | |
| B13681 | Ao-pPKS3-OMT4 | Cortinarius sp. KIS-3 PKS | This work |
| | | 1168005 (PKS3) and Cortinarius | |
| | | sp. KIS-3 OMT 1127603 (OMT4) | |
| | | under SES promoters (flanks to A. | |
| | | ory amyA locus) | |
| B13682 | Ao-pPKS3-OMT5 | Cortinarius sp. KIS-3 PKS | This work |
| | | 1168005 (PKS3) and C. | |
| | | semisanguineus OMT | |
| | | VTT_DN1090_OMT (OMT5) under | |
| | | SES promoters (flanks to A. ory | |
| | | amyA locus) | |
| B13693 | Tr-pPKS3-OMT4 | Cortinarius sp. KIS-3 PKS | This work |
| | | 1168005 (PKS3) and Cortinarius | |
| | | sp. KIS-3 OMT 1127603 (OMT4) | |
| | | under SES promoters (flanks to T. | |
| | | reesei CBH2 locus) | |
| B13694 | Tr-pPKS3-OMT5 | Cortinarius sp. KIS-3 PKS | This work |
| | | 1168005 (PKS3) and C. | |
| | | semisanguineus OMT | |
| | | VTT_DN1090_OMT (OMT5) under | |
| | | SES promoters (flanks to T. | |
| | | reesei CBH2 locus) | |
| B13696 | YL_pPKS3-OMT5 | Cortinarius sp. KIS-3 PKS | This work |
| | | 1168005 (PKS3) and C. | |
| | | semisanguineus OMT | |
| | | VTT_DN1090_OMT (OMT5) under | |
| | | SES promoters (flanks to Ant | |
| | | locus) | |
| B13814 | Af_afr2 | Intermediate plasmid having | This work |
| | | flanks to A. fumigatus afr2 locus | |
| B13815 | Aoch_laeA | Intermediate plasmid having | This work |
| | | flanks to A. ochraceus laeA locus | |
| B13990 | Af_OMT2 | Cortinarius sp. KIS-3 OMT | This work |
| | | 1191744 (OMT2) under SES | |
| | | promoter targeted to A. | |
| | | fumigatus afr2 locus | |
| B13991 | Af_OMT5 | C. semisanguineus OMT | This work |
| | | VTT_DN1090_OMT (OMT5) under | |
| | | SES promoter targeted to A. | |
| | | fumigatus afr2 locus | |
| B13992 | Aoch_OMT4 | Cortinarius sp. KIS-3 OMT | This work |
| | | 1127603 (OMT4) under SES | |
| | | promoter targeted to A. | |
| | | ochraceus laeA locus | |
| B14387 | 2u_NAT_inter | Multi copy plasmid with | This work |
| | | nourseothricin resistance marker | |
| | | NAT | |
| B14443 | pSc_OMT2 | Cortinarius sp. KIS-3 OMT | This work |
| | | 1191744 (OMT2) under SES | |
| | | promoters with targeting arms to | |
| | | the S. cerevisiae HIS locus | |
| B14444 | pSc_OMT4 | Cortinarius sp. KIS-3 OMT | This work |
| | | 1127603 (OMT4) under SES | |
| | | promoters with targeting arms to | |
| | | the S. cerevisiae HIS locus | |
| B14445 | pSc_OMT5 | C. semisanguineus OMT | This work |
| | | VTT_DN1090_OMT (OMT5) under | |
| | | SES promoters with targeting | |
| | | arms to the S. cerevisiae HIS | |
| | | locus | |
| B14909 | pNt-OMT4 | N-terminal his tagged OMT4 | This work |
| | | (KIS3_OMT_1127603) expressed | |
| | | from 2 micron plasmid with NAT | |
| | | selection marker | |
| B14911 | pNt-OMT5 | N-terminal his tagged OMT5 | This work |
| | | (VTT_DN1090_OMT) expressed | |
| | | from 2 micron plasmid with NAT | |
| | | selection marker | |
| B14912 | pNt-OMT2 | N-terminal his tagged Cortinarius | This work |
| | | sp. KIS-3 OMT 1191744 (OMT2) | |
| | | expressed from 2 micron plasmid | |
| | | with NAT selection marker | |

### Example 6

Construction of Saccharomyces cerevisiae Strains

To construct the NpgA-ACC1** base strain (H6108; Table 2), the plasmid B13474 (Table 1) was digested with NotI and the integration cassette was transformed to H4590 strain (Table 2) using LiAc and PEG mediated transformation (Gietz and Woods, 2006). The transformed strain was plated on SC-LEU selection plates and tested for the correct genomic integrations into the LEU loci using PCR.

The resulting H6108 strain (Table 2) was used as parental strain for all the other constructs. Thus, this strain was transformed separately with the PKS3, PKS3-OMT2, PKS3-OMT4, or PKS3-OMT5 expression cassettes (released from the B13477, B13480, B13482, and B13483 plasmids [Table 1], respectively, by NotI digestion) and the transformants were selected on SC^{-HIS} plates. The transformants were screened for the presence of the expression cassette and deletion of the HIS3 locus using qPCR.

The decarboxylase expression cassette was transformed to PKS3-OMT4 strain (H6117; Table 2) using the CRISPR-Cas9 method. The pLL164 Cas9-gRNA plasmid (Table 1) along with the B13485-NotI digested repair fragment was transformed to this strain and selected on YPD+NAT (nourseothricin, 200 ug/mL) plates. The transformed colonies were later verified by PCR for the presence of the expression cassette and the deletion of the target loci.

TABLE 2
Saccharomyces cerevisiae strains used in this study.

| | Parental | | |
|---|---|---|---|
| Number | strain | Additional modification to parental strain | Reference |
| H3900 | CEN.PK102- | ura3-52, his3-delta1, leu2-3, 112, TRP1, | VTT depository |
| | 5B | MAL2-8c, SUC2 | |
| H4590 | H3900 | ura3 :: (BM3R1-VP16AD sTF + URA3), his3- | VTT depository |
| | | delta1, leu2-3, 112, TRP1, MAL2-8c, SUC2 | |
| H6108 | H4590 | leu2 :: (npgA-acc1** + KlLEU), his3-delta1 | This work |
| H6111 | H6108 | his3 :: (PKS3 + Sp-HIS) | This work |
| H6115 | H6108 | his3 :: (PKS3- OMT2 + Sp-HIS) | This work |
| H6117 | H6108 | his3 :: (PKS3- OMT4 + Sp-HIS) | This work |
| H6118 | H6108 | his3 :: (PKS3- OMT5 + Sp-HIS) | This work |
| H6163 | H6117 | xii-4 :: DC | This work |

### Example 7

Construction of Yarrowia lipolytica Strains

Y. lipolytica transformation was carried out using the EZ Yeast transformation kit (Zymo Research). The H5887 strain (Table 4) was grown to OD 2 in 50 mL YPD medium at 30 °C. The PKS3-OMT5 cassette was obtained by digesting B13696 plasmid (Table 1) with NotI and was transformed according to the manufacturer's protocol. The transformants were then plated on selective YPD+NAT (200 μg/mL) plates for two days. Colonies were screened using PCR to detect the presence of the expression cassettes integrated into the genome.

TABLE 3
List of Yarrowia lipolytica strains used in this study.

| | Parental | Additional modification to | |
|---|---|---|---|
| Number | strain | parental strain | Reference |
| H5887 | C-00365 (BEL | intD1 :: npgA | VTT |
| | BF9/A-9) | | depository |
| H6223 | H5887 | ant :: L-SES(Yl_ 565 - Yl- | This work |
| | | 242cp)_(BnTAFM)_PKS3 + | |
| | | OMT5 | |

### Example 8

Construction of Aspergillus oryzae and Trichoderma reesei Strains:

A. oryzae M4993 and T. reesei M2157 (Table 3) were grown on potato dextrose agar plates supplemented with 1M sorbitol and 5 mM uridine for 4-5 days at 28 °C. Spores were collected into 2 mL glycerol stock solution (20% glycerol, 0.8% NaCl and 0.025% Tween-20) and were used to inoculate 300 mL YP+3% gelatin media supplemented with 10 mM uridine, and cultured at 28 °C for 16 hours with shaking at 200 rpm. Mycelium was collected by filtering the culture through a layer of sterile Miracloth (Calbiochem, Germany). The collected mycelium was washed first with sterile water and then with cold KMC (1 M KCl, 25 mM CaCl₂), 10 mM Tris-HCl, pH 5.8) buffer. The filtered mycelia were resuspended in the lytic solution (20 mg/mL Trichoderma harzianum lysing enzyme in 20 mL KMC buffer for A. oryzae, and 10 mg/mL yatalase in 10 mL KMC for T. reesei) and incubated at 30 °C with shaking at 80 rpm for 3 hours. The resulting protoplast solutions were filtered through a sterile cotton plug filter. The filtered protoplast solutions were centrifuged and washed with cold KMC buffer, followed by final washing with STC solution (1.33 M sorbitol, 10 mM Tris-HCl, 50 mM CaCl₂), pH 8.0). All the centrifugation steps were done for 4 min at 1500 rpm at 4 °C. After washing, protoplasts were resuspended in STC to a total volume of about 400 μL.

The PKS3-OMT4 or the PKS3-OMT5 expression cassettes were obtained by digesting the corresponding plasmids with MssI (Table 1). The digested plasmids were mixed with 100 μL of protoplast solution and incubated with 100 μL of transformation solution (25% PEG 6000, 50 mM CaCl₂), 10 mM Tris-HCl, pH 7.5) on ice for 20 min. Two mL of the transformation solution was then separately added to the protoplasts and the mixtures were incubated at room temperature for 5 min. The transformation mixtures were then diluted with STC, mixed with 7 mL of molten TOP agar and poured on the selection plates. The plates were incubated for 3 days, and the colonies were then replated on selection plates followed by screening using PCR.

TABLE 4
Aspergillus oryzae and Trichoderma reesei
strains used in this study.

| | Parental | Additional modification to parental | |
|---|---|---|---|
| Number | strain | strain | Reference |
| M2157 | — | Δpep1 Δtsp1 Δpep4 Δgap1 Δslp1 Δpep3 | VTT |
| | | Δpep5 Δpep2 Δamp2 Δslp8 Δsep1 Δpyr4 + | depository |
| | | Tr119989cp (CP2)-Bm3R1-VP16 | |
| M4993 | — | ΔprtT Δlig4 ΔoryA ΔpepC ΔpepY | VTT |
| | | ΔpepM1 ΔpyrG + 2x sTF (BnTAF1M) | depository |
| M6575 | M4993 | pyrG :: (PKS3-OMT4 + A. niger pyrG) | This work |
| M6576 | M4993 | pyrG :: (PKS3-OMT5 + A. niger pyrG) | This work |
| M6577 | M2157 | pyr4 :: (PKS3-OMT4 + pyr4) | This work |
| M6578 | M2157 | pyr4 :: (PKS3-OMT5 + pyr4) | This work |

### Example 9

Strain Cultivation and Sample Preparation: S. cerevisiae and Y. lipolytica

Three positive transformants for each strain were cultivated in 4 mL YP (20 g peptone, 10 g yeast extract per one liter water)+4% dextrose and/or TSB (17 g Tryptone, 3 g Soytone, 5 g NaCl, 2.5 g K₂HPO₄ per one liter water, pH 7.3)+2% glucose medium in 24-well plates at 28 °C for 72 hours with shaking at 200 rpm. Cultures were then centrifuged, and supernatants were collected and analysed by LC-MS. For extraction of compounds from the cell pellet, the cells were incubated in 1 mL MeOH at 37 °C with shaking at 240 rpm for 1 hour. The mixtures were centrifuged at 4000 rpm for 5 min, the supernatants were collected, and these MeOH extracts were analysed by LC-MS.

### Example 10

Strain Cultivation and Sample Preparation: A. Oryzae and T. reesei

Ten transformants for each strain were cultivated in 4 mL BMDY media (10 g Yeast extract, 20 g Bacto peptone, 0.4 mg Biotin, 20 g glucose, 31 g Ca₃(PO₄)₂, 13.4 g Yeast Nitrogen base with ammonium sulphate without amino acids per one liter water) in 24-well plates at 28 °C with shaking at 200 rpm for 5 days. Supernatant was collected by centrifugation and used directly for analysis. For extraction of compounds from the cells, mycelia were collected and resuspended in 1 mL MeOH. 500 μL of glass beads were added and cell lysis was performed by vortexing twice at 6500 rpm for 30 seconds. Samples were then centrifuged, and the supernatant was then collected for LC-MS analysis.

### Example 11

### Strain Cultivation and Sample Preparation: Biotransformation

For the biotransformation experiments, emodin (Sigma) was dissolved in ethanol and fed to the S. cerevisiae or Y. lipolytica cultures. Cultivations were done in 50 mL YP+2% dextrose, supplemented with 50 mg/L emodin, at 28 °C for 72 hours with shaking at 240 rpm. Supernatant samples and methanol cell extracts were collected for the analysis as described previously.

### Example 12

### LC-MS Method

Analysis was performed on an Acquity UHPLC system, Waters (Milford, MA, USA) and Synapt G2-S MS system, Waters (Milford, MA, USA). Chromatography was performed using an ACQUITY UPLC BEH HSS T3 column, 1.8 μm 2.1×100 mm, (Waters), kept at 45 °C. The experiment was carried out at a flow rate of 0.4 ml/min with mobile phase A (0.1% formic acid in water) and B (0.1% formic acid in acetonitrile). The gradient elution started at 5% B maintained for 0.2 min, then increased to 90% B within 14 min, then directly returned to 5% B and maintained for 3 min. Mass spectrometry was carried out by electrospray ionization (ESI) in negative polarity using the capillary voltage at 2.0 kV and cone voltage kV. Source temperature was 150 °C and desolvation temperature was 500 °C. The desolvation gas flow was set at 800 L/h (nitrogen).

### Example 13

Sequencing the Genomic DNA of Cortinarius semisanguineus

A pure culture of Cortinarius semisanguineus was obtained from the Natural Resources Institute Finland (Luke). The mycelium was grown at 20 °C on plates with a cellophane film on top of a modified Melin-Norkrans agar medium with reduced sugar content (½MMN). ½MMN agar medium contains 1.25 g glucose, 5 g malt extract, 0.25 g (NH₄)₂HPO₄, 0.5 g KH₂PO₄, 0.15 g MgSO₄*7H₂O, 0.05 g CaCl₂)*2H₂O, 0.025 g NaCl, 20.16 mg Fe-EDTA, 10 mg thiamine-HCl and 18 g of agar per one liter water, and pH value was set at 5.5.

Mycelium grown on cellophane film was collected, frozen and ground in liquid nitrogen. The genomic DNA was isolated and purified with QIAGEN Genomic-tip 500/G columns according to the manufacturer's protocol. Still another purification step was performed by adding 600 μl of buffer (200 mM Tris-HCl pH 8.5, 250 mM NaCl, 25 mM EDTA pH 8.0 and 0.5% SDS) and 1 volume of phenol-chloroform-isoamyl alcohol (ratio 25:24:1) and mixing for 10 min with a rotator shaker at room temperature. After centrifuging 1 h at 13500 rpm, the water-phase was collected. Extraction with phenol-chloroform-isoamyl alcohol was repeated twice followed by extraction with chloroform-isoamyl alcohol (ratio 24:1) twice. The DNA was precipitated from the water-phase at room temperature with 0.54 volumes of isopropanol and centrifuged for 2 min at 13500 rpm. The DNA was washed with 70% ethanol and dried before dissolving in 10 mM Tris-HCl pH 8.

The sample was sent to Novogene (UK) for Fungus Genome Fine Mapping. After sample quality check, microbial whole genome libraries were prepared and Illumina NovaSeq PE150 and Pacbio sequencing were performed. The aim was 70× read coverage depth with 5 Gb sequencing but because of very high repeat rate of the genome only 2.827 Gb of high quality nonrepetitive reads could be obtained even after 10 Gb sequencing.

### Example 14

Sequencing mRNA from Cortinarius semisanguineus

RNA was extracted from three types of C. semisanguineus tissues that were expected to have a different transcription profile of the genes involved in pigment formation; white mycelium from pure in vitro culture and a sample from a yellow stipe and a blood-red cap from a fungal fruiting body. Pure culture of C. semisanguineus was grown on ½MMN liquid medium as a static culture where the mycelium was growing on the air-liquid interface. The fungal fruiting body was collected from pine forest at Southern Finland and stored frozen-20 °C for 8 months before RNA extraction. The pure culture did not originate from the same fruiting body as the cap and stipe. All these three tissues in two parallels were first ground in liquid nitrogen and the total RNA was extracted with Qiagen RNeasy Mini Kit according to the manufacturer's protocol and eluted with RNAse-free water.

The samples were sent to Novogene (UK) for “Plant and animal whole genome sequencing” (WOBI) where after a sample quality check an mRNA library was prepared with poly-A enrichment and Illumina NovaSeq PE150 sequencing was performed, resulting in 6 G of raw data (20 M reads) per sample.

### Example 15

Bioinformatic Identification of the Anthraquinone Biosynthetic Gene Clusters of Cortinarius sp. KIS-3 and Cortinarius semisanguineus

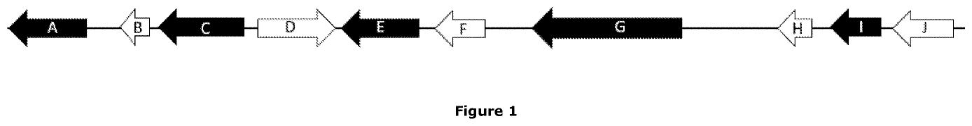

To identify the potential biosynthetic gene cluster responsible for anthraquinone pigment production in Cortinarius sp. KIS-3 and C. semisanguineus, a comparative genomics approach was used. Using the already published C. odorifer nrPKS (Löhr et al. 2022) as the bait sequence, we identified two nrPKS candidates each from Cortinarius sp. KIS-3 (FIG. 1 and Table 5) and C. semisanguineus (Table 5). The high level expression of the C. semisanguineus PKS1 and PKS2 biosynthetic genes in the stipe and the cap where anthraquinone pigment production is active, and the repressed expression of this cluster in the pure in vitro culture where anthraquinone production is missing, were verified from the transcriptomic datasets.

TABLE 5
Potential nrPKSs in Cortinarius species identified in this study.

| PKS | Short name | Source |
|---|---|---|
| PKS 1168005 | PKS3 | Cortinarius sp. KIS-3 |
| PKS 856087 | PKS4 | Cortinarius sp. KIS-3 |
| VTT_DN492-683_PKS | PKS1 | C. semisanguineus |
| VTT_DN14_PKS | PKS2 | C. semisanguineus |

### Example 16

Functional Identification of PKS3 from Cortinarius sp. KIS-3

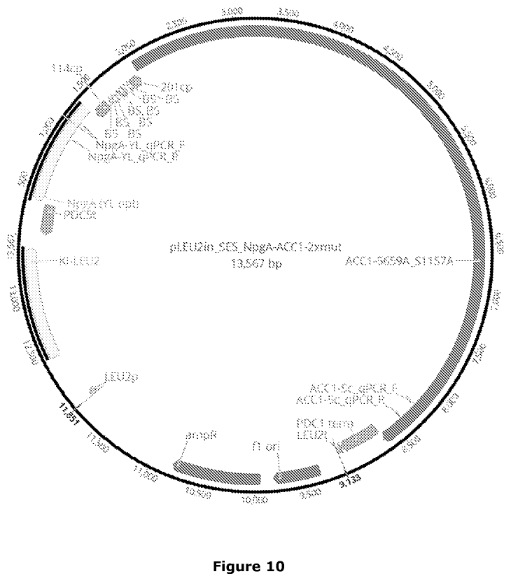

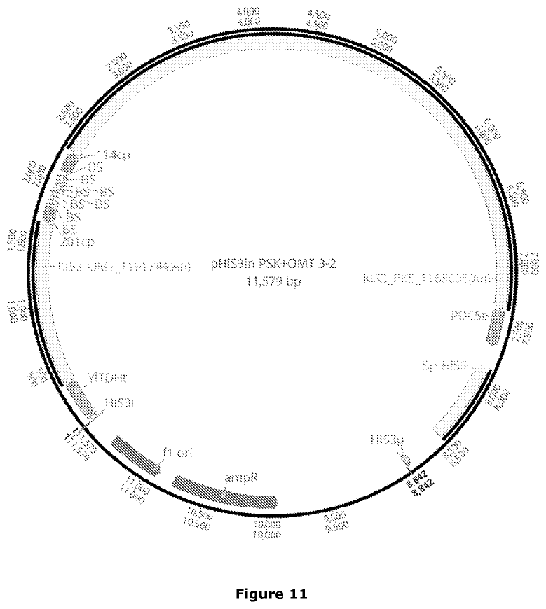

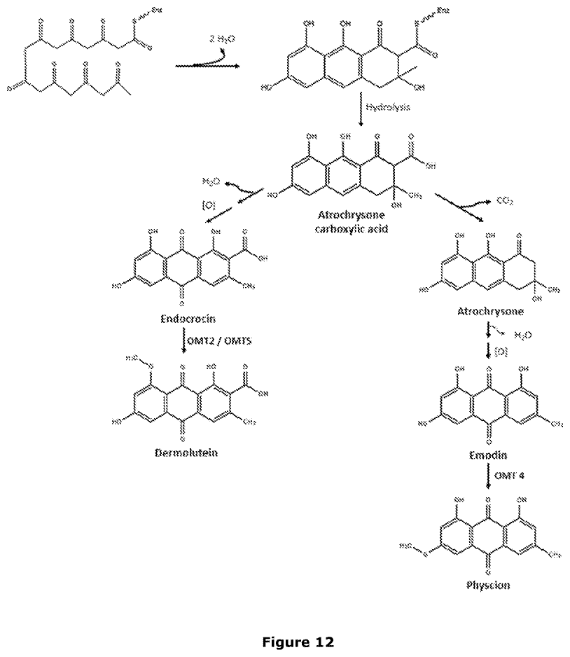

For the functional expression of potential PKS candidates (Table 6) in S. cerevisiae, the genes encoding the phosphopantetheinyl transferase NpgA (essential for PKS activity) and ACC1** (to increase the supply of anthraquinone precursor malonyl-CoA) were successfully integrated into the LEU locus of S. cerevisiae (FIG. 10) to construct the base strain H6108 (Table 2). This strain was then further transformed by introducing PKS expression cassettes into the HIS locus of S. cerevisiae.

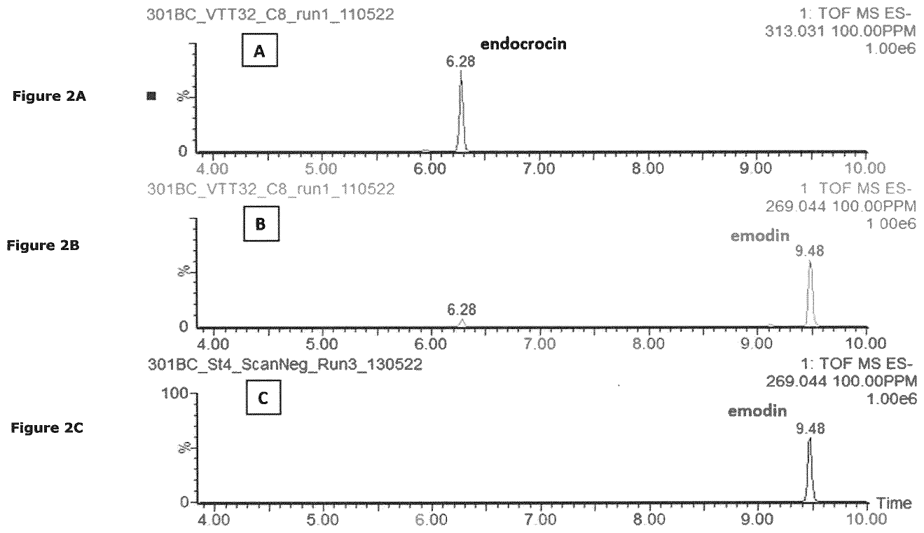

Cultivation of these transformed S. cerevisiae strains in YPD (Yeast Peptone Dextrose media) and TSB (Tryptic Soy Broth) and SCD (Synthetic Complete Dextrose) media led to the identification of the Cortinarius sp. KIS-3 PKS 1168005 (PKS3) as the polyketide synthase which produces the anthraquinone intermediates emodin and endocrocin (FIGS. 2A-2C).

### Example 17

Production of Emodin and Endocrocin in Other Fungal Hosts with Cortinarius sp. KIS-3 PKS 1168005 (PKS3)

Because the functionality of the SES expression system utilized in the expression cassettes described in Table 1 is independent of the identity of the fungal host strains, the same expression cassettes were also introduced to various additional fungal hosts.

Introduction of the Cortinarius sp. KIS-3 PKS 1168005 (PKS3) to Y. lipolytica H5887 and A. oryzae M4993 led to the production of the anthraquinone compounds emodin and endocrocin. In a similar way, T. reesei could also be used as a potential host for the production of these compounds.

### Example 18

### Utilization of a Decarboxylase to Increase Emodin Production

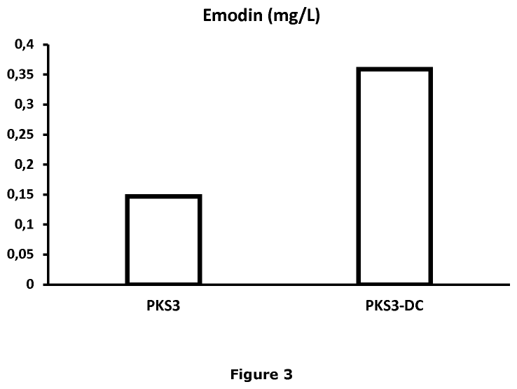

Introduction of the A. fischeri decarboxylase to the PKS3-expressing strain of S. cerevisiae led to an increase in the amount of emodin produced by the strain (FIG. 3). The decarboxylase enhances the decarboxylation of atrochrysone carboxylic acid, thus improving the overall productivity of emodin.

### Example 19

Identification of Anthraquinone O-Methyltransferases from Cortinarius sp. KIS-3 and Cortinarius semisanguineus

To further characterize the biosynthetic pathway from endocrocin towards dermolutein, and from emodin towards physcion, several O-methyltransferase (OMT) candidates were investigated. OMT2 of Cortinarius sp. KIS-3 (JGI MycoCosm 1191744) is encoded in the biosynthetic gene cluster that encodes PKS 856087 (PKS4). OMT5 of Cortinarius semisanguineus (VTT_DN1090_OMT) is encoded in the biosynthetic gene cluster that also encodes VTT_DN14_PKS (PKS2). OMT4 of Cortinarius sp. KIS-3 (JGI MycoCosm 1127603) is encoded in the biosynthetic gene cluster that also encodes PKS 1168600.

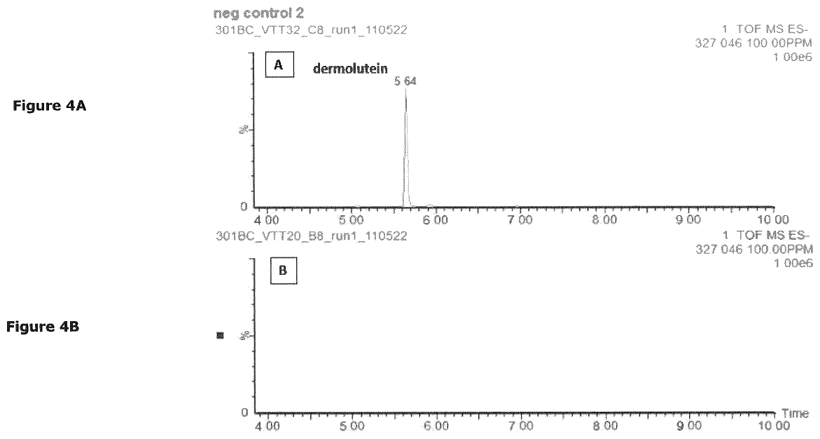

Expression of the OMT2 or OMT5 genes together with PKS3 in the S. cerevisiae H6108 strain (Table 2) led to the production of dermolutein (FIGS. 4A and 4B). The PKS3-OMT5 expression cassette was also successfully integrated to A. oryzae M4993 to construct strain M6576 that produces dermolutein. This demonstrates that the OMT2 from Cortinarius sp. KIS-3 and OMT5 from C. semisanguineus are the enzymes that carry out the O-methylation of endocrocin at the 8-OH group to yield dermolutein.

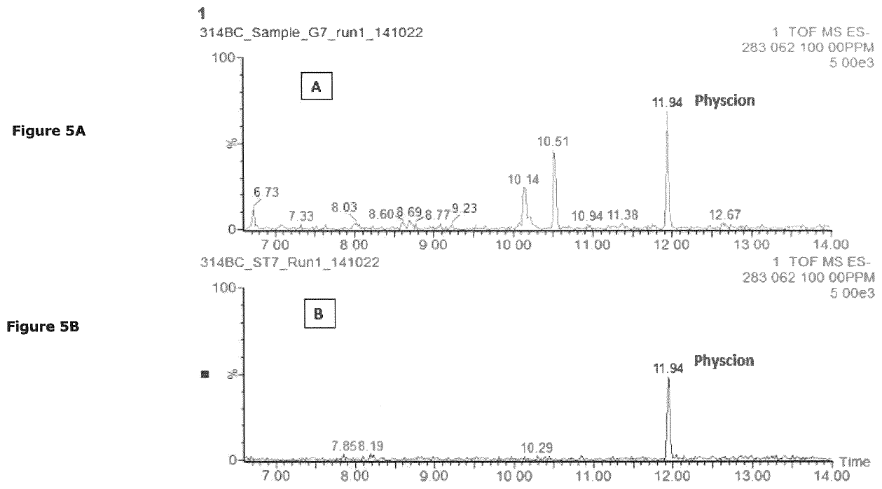

In addition, co-expression of the OMT4 gene of Cortinarius sp. KIS-3 with PKS3 in S. cerevisiae, and separately in A. oryzae as the host strains led to the production of physcion. Expression of the OMT4 gene in S. cerevisiae and Y. lipolytica hosts similarly allowed the biotransformation of externally supplied emodin to physcion. These results demonstrate that the OMT4 gene of Cortinarius sp. KIS-3 catalyzes the O-methylation of emodin at the 6-OH group to yield physcion (FIGS. 5A and 5B).

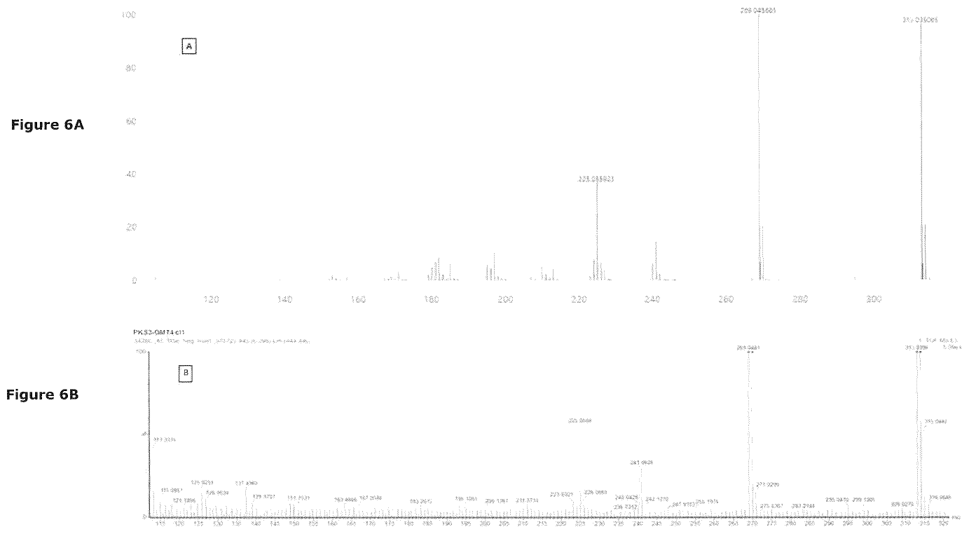

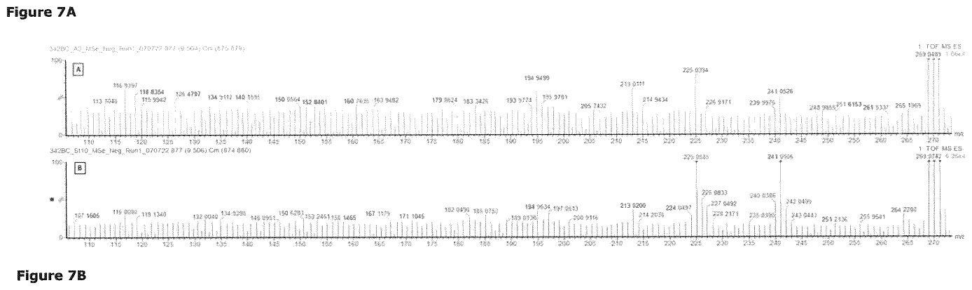

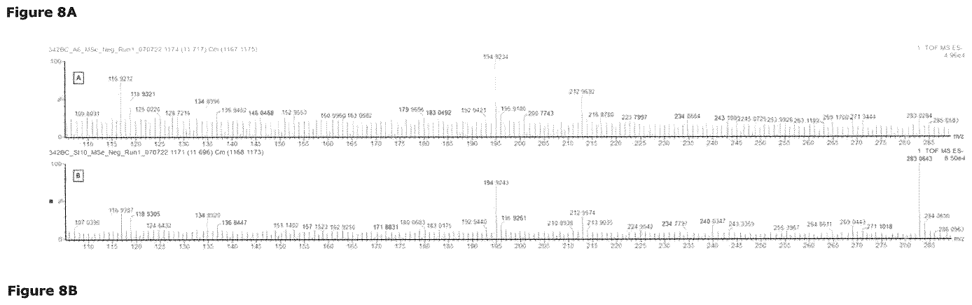

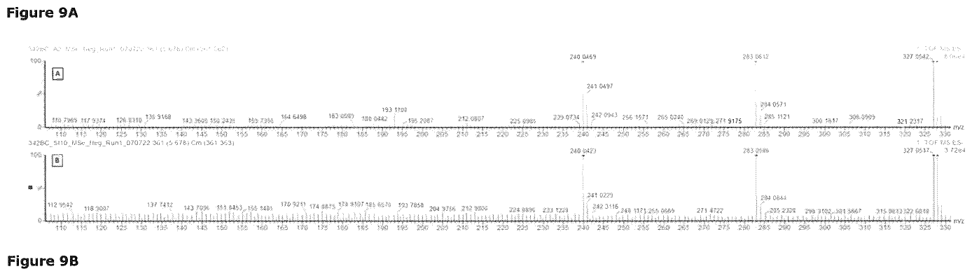

The identities of these compounds were confirmed using LC-MS/MS, by comparing the fragmentation patterns with the standards or data available from literature (FIGS. 6-9), and the percentage ppm error was found to be within the permissible range of ±20 ppm (Table 6).

TABLE 6
Percentage error in mass measured using LC-MS/MS.

| Compound | Theoretical m/z | Measured m/z | % Error (in ppm) |
|---|---|---|---|
| Endocrocin | 313.0348 | 313.0396 | −15.2379 |
| Emodin | 269.045 | 269.0489 | −14.4957 |
| Physcion | 283.0607 | 283.062 | −4.7693 |
| Dermolutein | 327.0505 | 327.0542 | −11.3744 |

### Example 20

Production of O-Methylanthraquinones with Recombinant S. cerevisiae Strains Expressing O-Methyltransferases from Cortinarius KIS-3 and Cortinarius Semisanguineus

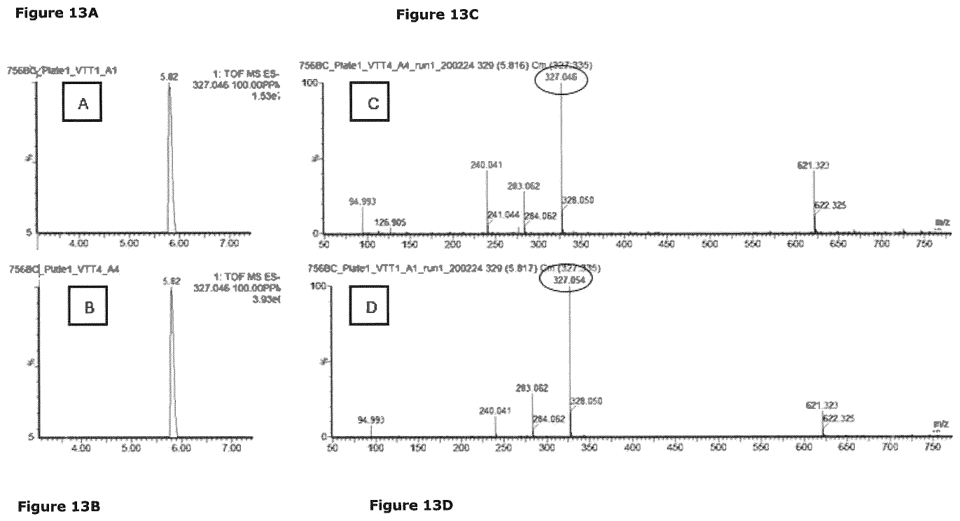

The genes encoding OMT2 (JGI MycoCosm 1191744) and OMT5 (VTT_DN1090_OMT) were inserted separately into the B13473 expression vector to produce expression vectors B14443 and B14445, respectively. These expression vectors were separately transformed into the S. cerevisiae H6108 host to integrate the expression cassettes into the HIS3 genomic locus. The resulting recombinant S. cerevisiae strains were grown for 72 hours with shaking at 220 rpm at 23 °C in TSB medium supplemented with 5 mM purified endocrocin. The cells were separated by centrifugation at 4,000 rpm for 5 min, and the supernatants were extracted twice with ethyl acetate. The organic fractions were collected, evaporated under reduced pressure, and the resulting extracts were dissolved in methanol. The methanol extracts were analyzed by LC-MS, and dermolutein was identified by comparison of its chromatographic mobility, UV spectrum, and MS/MS spectra with those of an authentic dermolutein standard (FIG. 13).

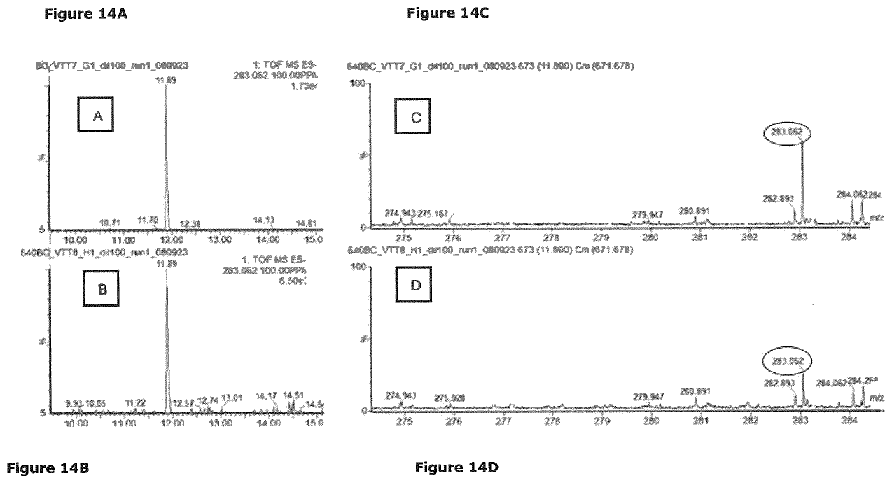

In addition, the gene encoding OMT4 (JGI MycoCosm 1127603) was inserted into the B13473 expression vector to produce expression vector B14444. This expression vector was transformed into S. cerevisiae H6108 host to integrate the expression cassette into the HIS3 genomic locus. The resulting recombinant S. cerevisiae strain was grown at 23 °C for 72 hours with shaking at 220 rpm in TSB medium supplemented with 5 mM purified emodin. The cells were separated by centrifugation at 4,000 rpm for 5 min, and the cells were extracted with methanol. The extracted cells were removed by centrifugation at 4,000 rpm for 5 min, and the resulting methanol fraction was collected. The methanol extract was analyzed by LC-MS, and physcion was identified by comparison of its chromatographic mobility, UV spectrum, and MS/MS spectra with those of an authentic physcion standard (FIG. 14).

### Example 21

Production of O-Methylanthraquinones with Anthraquinone-Producing Aspergillus Strains Expressing O-Methyltransferases from Cortinarius KIS-3 and Cortinarius Semisanguineus

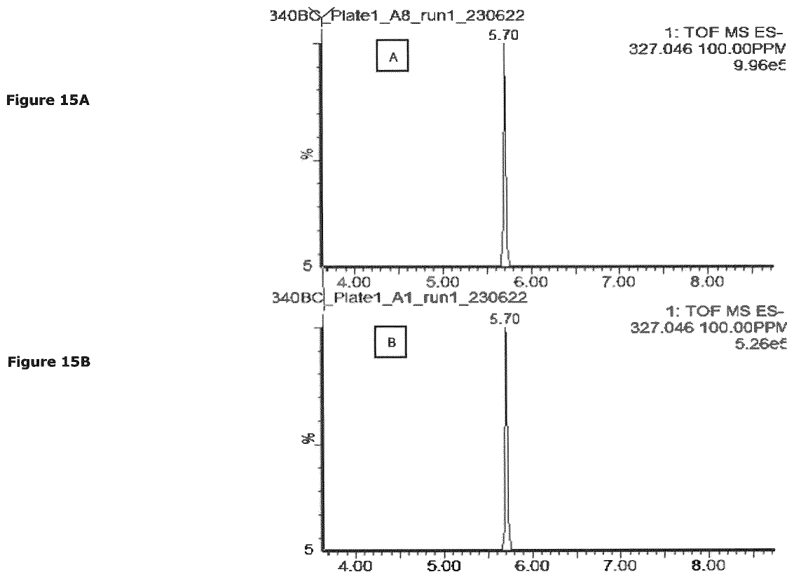

The genes encoding OMT2 (JGI MycoCosm 1191744) and OMT5 (VTT_DN1090_OMT) were inserted separately into the B13814 expression vector to produce expression vectors B13990 and B13991, respectively. These expression vectors were separately transformed into Aspergillus fumigatus, a natural producer of endocrocin (Lim et al. 2012), to integrate the expression cassettes into the genome of the host. The resulting recombinant A. fumigatus strains were grown at 29 °C for 10 days on solid GMM medium. The cultures were homogenized and extracted twice with ethyl acetate. The organic fractions were collected, filtered through a filter paper, evaporated under reduced pressure, and the resulting extracts were dissolved in methanol. The methanol extracts were analyzed by LC-MS, and dermolutein was identified by comparison of its chromatographic mobility, UV spectrum, and MS/MS spectra with those of an authentic dermolutein standard (FIG. 15).

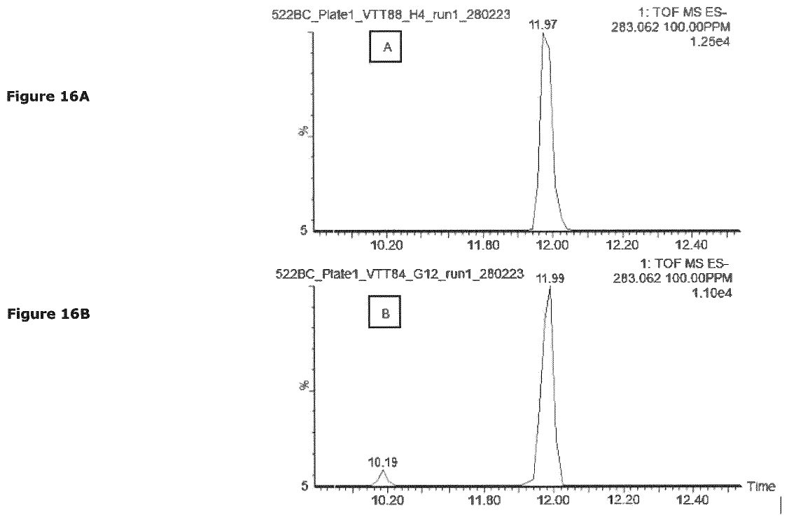

In addition, the gene encoding OMT4 (JGI MycoCosm 1127603) was inserted into the B13815 expression vector to produce expression vector B13992. This expression vector was transformed into Aspergillus ochraceus, a natural producer of emodin (Lu et al. 2010), to integrate the expression cassette into the genome of the host. The resulting recombinant A. ochraceus strain was grown at 32 °C for 120 hours in Czapek-Dox medium with shaking at 220 rpm. The mycelia were collected by centrifugation at 3,500 rpm for 10 min, the supernatant was discarded, and the mycelia were extracted twice with methanol. The organic fractions were collected, filtered through a filter paper, evaporated under reduced pressure, and the resulting extracts were dissolved in methanol. The methanol extracts were analyzed by LC-MS, and physcion was identified by comparison of its chromatographic mobility, UV spectrum, and MS/MS spectra with those of an authentic physcion standard (FIG. 16).

### Example 22

Production of O-Methylanthraquinones with Isolated Recombinant O-Methyltransferases from Cortinarius KIS-3 and Cortinarius semisanguineus

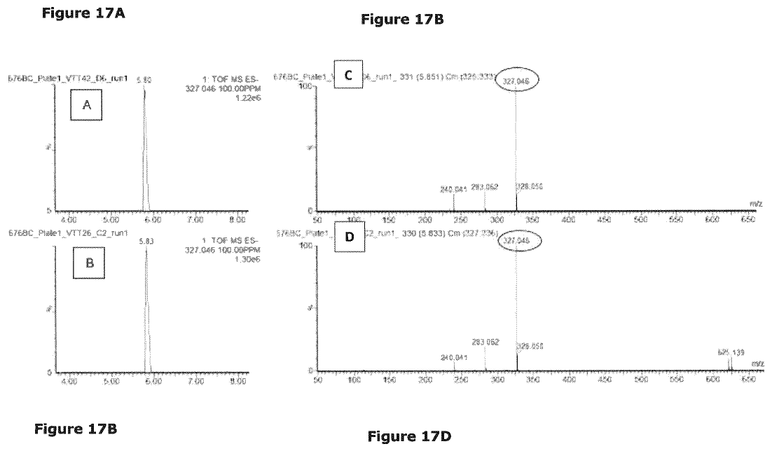

The genes encoding OMT5 and OMT2 were tagged with a His₆ tag at their 5′ ends and inserted separately into the B14387 expression vector to produce expression vectors B14911 and B14912, respectively. These expression vectors were separately transformed into S. cerevisiae H6111 host. The resulting recombinant S. cerevisiae strains were grown at 30 °C in YPD medium for 24 hours with shaking at 220 rpm. The cells were collected by centrifugation at 4,000 rpm for 10 min, resuspended in 50 ml of ice-cold lysis buffer (Binding buffer: 20 mM sodium phosphate, 0.5 M NaCl, 20 mM imidazole, pH 7.4), supplemented with protease inhibitors (1×complete, EDTA-free Protease Inhibitor Cocktail, Roche). Lysis was performed using a BeadBeater homogenizer (Biospec) and the extent of the cell lysis was monitored using a microscope. The resulting cell lysates were centrifuged at 27,000 g for 45 min at 4 °C, and the resulting clarified lysates were loaded onto 5 mL HiTrap Chelating HP columns equilibrated with Binding buffer. The OMT2 and OMT5 enzymes were eluted with elution buffer (20 mM sodium phosphate, 0.5 M NaCl, 500 mM imidazole, pH 7.4), and fractions were analyzed by SDS-PAGE. The fractions containing purified OMT2 and separately, the fractions containing purified OMT5 enzymes were then used to methylate endocrocin at the 8-OH group to yield dermolutein in vitro, in reaction mixtures containing 2 μM enzyme, 100 UM purified endocrocin, 20 mM Tris-HCl buffer pH 7.0 and 1 mM S-adenosylmethionine (SAM). The dermolutein product was detected by LC-MS, and identified by comparison of its chromatographic mobility, UV spectrum, and MS/MS spectra with those of an authentic dermolutein standard (FIG. 17).

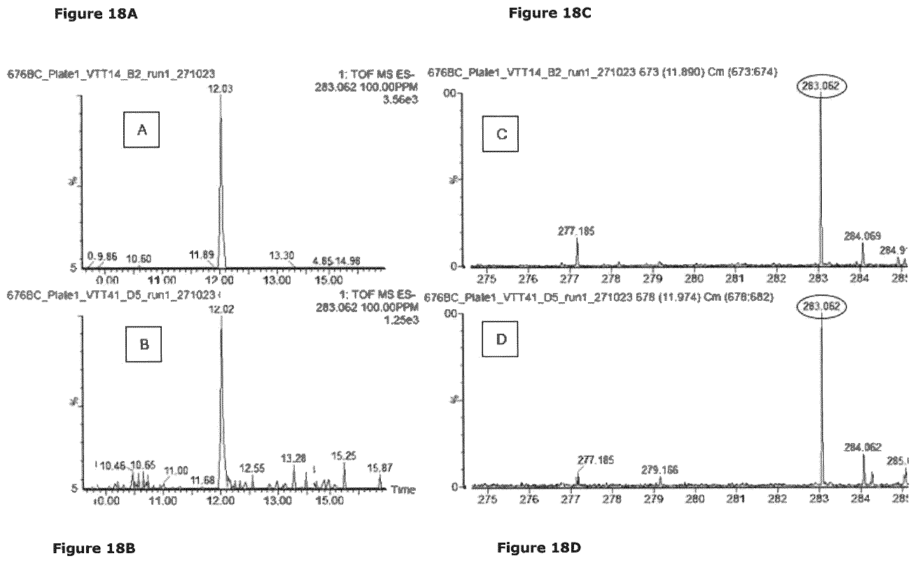

In addition, the gene encoding OMT4 was tagged with a His₆ tag at its 5′ end and inserted into the B13487 expression vector to produce expression vector B14909. This expression vector was transformed into S. cerevisiae H6111 host. The resulting recombinant S. cerevisiae strains were grown at 30 °C in YPD medium for 24 hours with shaking at 220 rpm. The cells were collected by centrifugation at 4000 rpm for 10 min, resuspended in 50 ml of ice-cold lysis buffer (Binding buffer: 20 mM sodium phosphate, 0.5 M NaCl, 20 mM imidazole, pH 7.4), and supplemented with protease inhibitors (1×complete, EDTA-free Protease Inhibitor Cocktail, Roche). Lysis was performed using a BeadBeater homogenizer (Biospec) and the extent of the cell lysis was monitored using a microscope. The resulting cell lysates were centrifuged at 27,000 g for 45 min at 4 °C, and the resulting clarified lysates were loaded onto 5 mL HiTrap Chelating HP columns equilibrated with Binding buffer. The OMT4 enzyme was with elution buffer (20 mM sodium phosphate, 0.5 M NaCl, 500 mM imidazole, pH 7.4), and fractions were analyzed by SDS-PAGE. The fractions containing purified target OMT4 were collected and then used to methylate emodin at the 6-OH group to yield physcion in vitro, in reaction mixtures containing 2 μM enzyme, 100 UM purified endocrocin, Tris-HCl buffer, and 1 mM S-adenosylmethionine (SAM). The physcion product was detected by LC-MS, and identified by comparison of its chromatographic mobility, UV spectrum, and MS/MS spectra with those of an authentic physcion standard (FIG. 18).

### Example 23

### Docking Models for the Cortinarius OMT-SAH-AQ Complexes

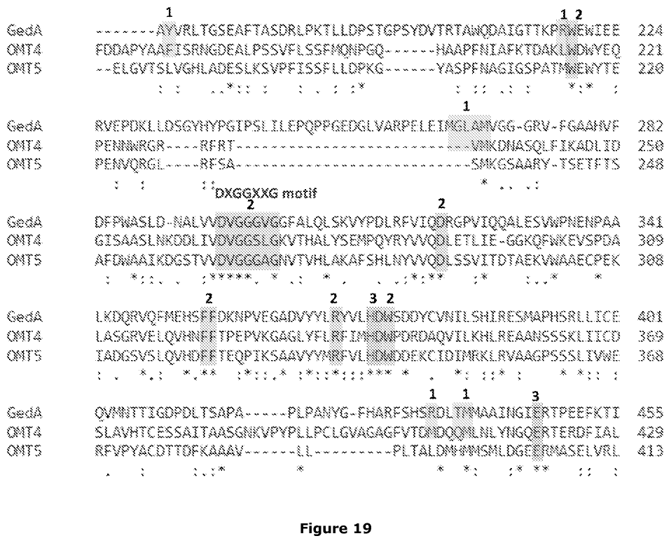

A sequence alignment of SEQ ID NO: 2 (OMT4), SEQ ID NO: 3 (OMT5), and GedA (the emodin-8-OMT of A. terreus, Xue et al. 2022), is presented in FIG. 19. The primary amino acid sequences of SEQ ID NO: 2 and SEQ ID NO: 3 show low level of identities (less than 30%) to GedA (PDB id: 7WH9, the emodin-8-OMT of A. terreus, Xue et al. 2022). Nevertheless, this alignment allows the initial assignment of residues for the SAM binding site, the substrate binding site and the catalytic active site of these enzymes, as indicated in FIG. 19.

To gain further insight into the structure/function relationships of these proteins, we generated AlphaFold protein structure models for SEQ ID NO: 1 (OMT2) and SEQ ID NO: 3 (OMT5). These two models were practically identical, with key amino acids (see below) completely conserved, in agreement with that the primary amino acid sequences of the two proteins are 90% identical. We have also generated an AlphaFold protein structure model for SEQ ID NO: 2 (OMT4).

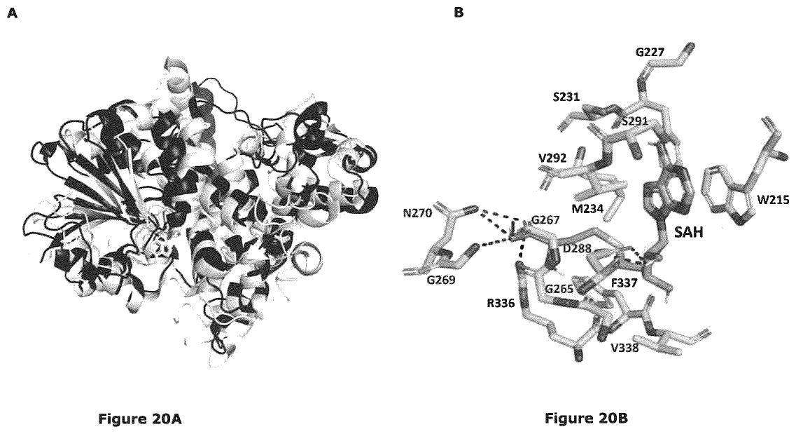

Our structure models for SEQ ID NO: 1 (OMT2) and SEQ ID NO: 3 (OMT5) aligned well with the recently published crystal structure of GedA (RMSD [Root mean square deviation] of 3.52 Å and 3.44 Å to OMT2 and OMT5, respectively) (FIG. 20A).

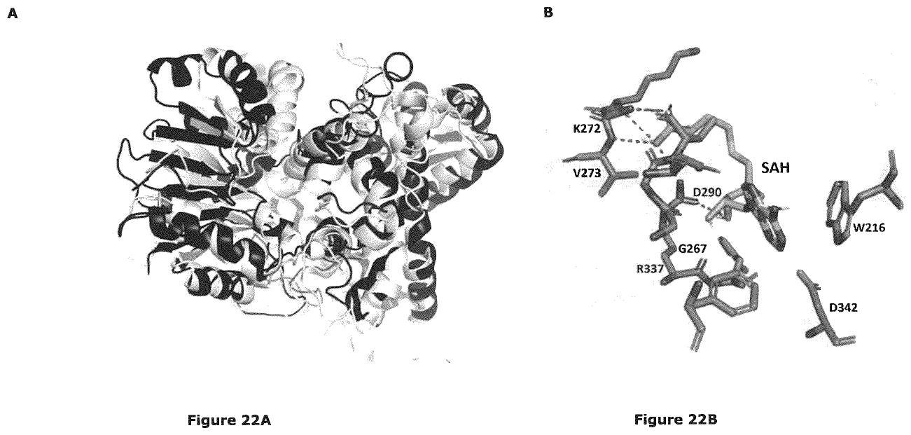

Our structure model for SEQ ID NO: 2 (OMT4) also aligned well with the recently published crystal structure of GedA (RMSD [Root mean square deviation] of 2.65) (FIG. 22A).

The co-product S-adenosylhomocysteine (SAH) was then docked into the SEQ ID NO: 1 and SEQ ID NO: 3 models to reveal identical SAH binding poses and SAM binding site residues. The SAH binding poses and the SAM binding site residues are also similar to those of GedA (FIG. 20B with predicted hydrogen bonds and key interactions indicated). In SEQ ID NO: 1 and SEQ ID NO: 3, aromatic residues W215 are predicted to interact with the adenine moiety of SAH through n-n stacking. The ribose moiety of SAH may be stabilized by the canonical DXGGXXG motif (FIG. 19) along with hydrogen bonds to D288. The homocysteine tail of SAH may also establish an extensive hydrogen bonding network with R336, G267, and N270.

The co-product S-adenosylhomocysteine (SAH) was also docked into the SEQ ID NO: 2 (OMT4) model to reveal a highly conserved SAM binding site, similar to that of GedA (FIG. 22B with predicted hydrogen bonds and key interactions indicated). Thus, the aromatic residue W216 of SEQ ID NO: 2 is predicted to interact with the adenine moiety of SAH through n-n stacking. The ribose moiety of SAH may be stabilized by the canonical DXGGXXG motif along with hydrogen bonds to R337. The homocysteine tail of SAH may also establish an extensive hydrogen bonding network with R337, K272, and V273.

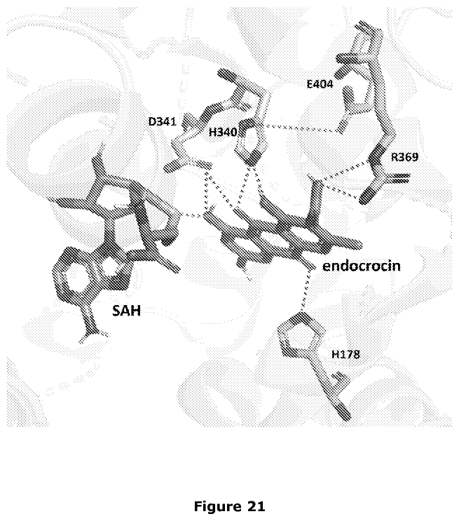

Next, the substrate endocrocin was docked into the SAH-OMT complex of SEQ ID NO: 1 and SEQ ID NO: 3. The predicted substrate binding sites in SEQ ID NO: 1 and SEQ ID NO: 3 (OMT2 and OMT5, completely overlapping models) are presented in FIG. 21. The co-product S-adenosylhomocysteine (SAH) and the substrate (endocrocin) are shown as well as the substrate binding site residues and catalytic residues of SEQ ID NO: 1 and SEQ ID NO: 3 (completely overlapping and identical residues). Furthermore, predicted hydrogen bonds and key interactions are also indicated in FIG. 21. These docked models revealed conserved active site residues which could contribute to the SN2-type nucleophilic reaction, as seen with typical class II OMTs including GedA (Xue et al. 2022). Thus, E404 may serve as an acid to polarize H340 which then acts as a base to deprotonate the 8-OH of endocrocin. The corresponding phenolate ion of the substrate would then act as nucleophiles to abstract the positively charged methyl group of SAM, giving rise to the O-methylated product dermolutein along with SAH.

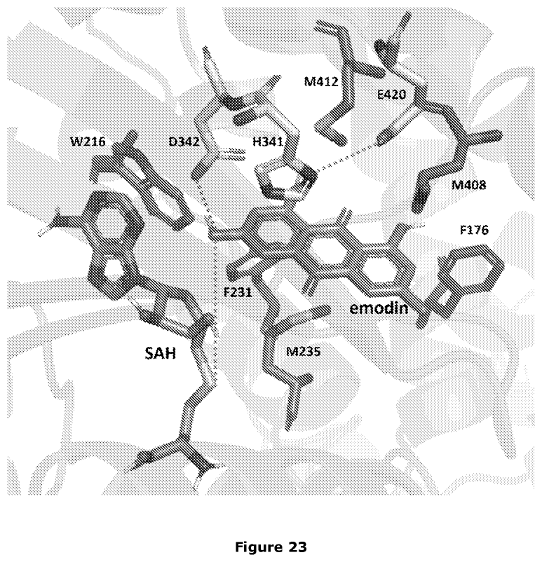

Next, the substrate emodin was docked into the SAH-OMT complex of SEQ ID NO: 2. The predicted substrate binding site in SEQ ID NO: 2 (OMT4) is presented in FIG. 23. The co-product S-adenosylhomocysteine (SAH) and the substrate (emodin) are shown as well as the substrate binding site residues and catalytic residues of SEQ ID NO: 2. Furthermore, predicted hydrogen bonds and key interactions are also indicated in FIG. 23. This docked model revealed conserved active site residues which could contribute to the SN2-type nucleophilic reaction, as seen with typical class II OMTs including GedA (Xue et al. 2022). Thus, E420 may serve as an acid to polarize H341 which then acts as a base to deprotonate the 6-OH of emodin. The corresponding phenolate ion of the substrate would then act as nucleophiles to abstract the positively charged methyl group of SAM, giving rise to the O-methylated product physcion along with SAH.

While the active site residues and the SAH binding sites are similar in SEQ ID NO: 1, SEQ ID NO: 3 and SEQ ID NO: 2, the predicted substate binding sites diverge between SEQ ID NO: 2 on one hand, and SEQ ID NO: 1 and SEQ ID NO: 3 on the other hand. The substrate binding sites of all three enzymes are also divergent from that of GedA. These distinct substrate binding cavities determine the different substrate selectivities and the different regioselectivities for O-methylation (emodin 8-OH is O-methylated by GedA; emodin 6-OH is O-methylated by SEQ ID NO: 2; and endocrocin 8-OH is O-methylated by SEQ ID NO: 1 and SEQ ID NO: 3).

In SEQ ID NO: 1 and SEQ ID NO: 3, the aromatic plane of the endocrocin substrate is bracketed by H178 and H395 from one side and D341 and H340 from the opposite side (FIG. 21 with predicted hydrogen bonds and key interactions indicated; the indicated residues are identical in the two sequences). These residue pairs form hydrogen bonds with the two carbonyl oxygens and stabilize the horizontal position of the substrate. Additionally, the carboxylic acid moiety of endocrocin establishes hydrogen bonds with the R369 side chain, thus prohibiting a 180° rotation of the substrate along to the plane. This ensures that the C8 instead of the C1 phenolic alcohol approaches the methyl group of SAM. Meanwhile, D341 forms hydrogen bonds with both the 8-OH and the C9 carbonyl group, breaking the intramolecular hydrogen bond in endocrocin and setting up 8-OH for methylation by SEQ ID NO: 1 and SEQ ID NO: 3 to yield dermolutein.

In SEQ ID NO: 2 (OMT4), emodin is surrounded by F231, F338, and M408 which contribute hydrophobic and Van der Waals interactions, while residues M235 and M412 stabilize the substrate from opposite sides of the aromatic plane (FIG. 23 with predicted hydrogen bonds and key interactions indicated). Residue D342 forms a hydrogen bond with the 6-OH group of emodin, orienting this moiety towards the methyl group of SAM for physcion formation by SEQ ID NO: 2. In GedA, this phenolic alcohol is engaged in hydrogen bonds with residues R434 and Y173, exposing the 8-OH group for methylation to yield questin. However, these GedA residues are replaced by M407 and F176 in SEQ ID NO: 2, thus a pose that would orient the 8-OH group of emodin towards the activated methyl group of SAM is not favored in SEQ ID NO: 2, thus SEQ ID NO: 2 does not produce questin, but yields physcion.

### REFERENCES

CN 108004193
Anderson, J. A. Conversion of emodin to physcion by a cell-free preparation of Aspergillus parasiticus. Phytochemistry, 1986. Vol 25, p. 1115-1117
Gietz R. D., Woods R. A. Yeast transformation by the LiAc/SS Carrier DNA/PEG method. Methods Mol Biol, 2006, 313:107-120. 10.1385/1-59259-958-3:107
Griffiths S. et al. Elucidation of cladofulvin biosynthesis reveals a cytochrome P450 monooxygenase required for anthraquinone dimerization. Proc Natl Acad Sci, 2016, 113, 6851-6856. 10.1073/pnas.1603528113.
Lim F. Y. et al. Genome-Based Cluster Deletion Reveals an Endocrocin Biosynthetic Pathway in Aspergillus fumigatus. Appl Environ Microbiol, 2012, 78. doi.org/10.1128/AEM.07710-11
Lu P., Zhao X., Cui T. Full Length Research Paper Production of emodin from Aspergillus ochraceus at preparative scale. Afr. J. Biotechnol. 2010; 9:512-517
Löhr, F. Eisen, W. Thiele, L. Platz, J. Motter, W. Hüttel, M. Gressler, M. Müller, D. Hoffmeister, Unprecedented Mushroom Polyketide Synthases Produce the Universal Anthraquinone Precursor Angew. Chem. Int. Ed. 2022, 61, e202116142; Angew, Chem. 2022, 134, e202116142.
Parajuli, P., Pandey, R. P., Nguyen, T. H. T. et al. Substrate Scope of O-Methyltransferase from Streptomyces peucetius for Biosynthesis of Diverse Natural Products Methoxides. Appl Biochem Biotechnol 184, 1404-1420 (2018). doi.org/10.1007/s12010-017-2603-4
Qi, F. et al. Microbial production of the plant-derived fungicide physcion, Metabolic Engineering, 2022, 74, pp. 130-138.
Räisänen R. Fungal colorants in applications—focus on Cortinarius species. Color Technol., 2018, 135, 22-31 8 Nov. 2018. 10.1111/cote.12376
Sun L. et al. Metabolic engineering of Saccharomyces cerevisiae for efficient production of endocrocin and emodin. Metabolic Engineering, 2019, 54, 212-221. 10.1016/j.ymben.2019.04.008
Xue Y. et al. Characterization and Structural Analysis of Emodin-O-Methyltransferase from Aspergillus terreus. Journal of Agricultural and Food Chemistry, 2022, Vol 70, p. 5728-5737. doi: 10.1021/acs.jafc.2c01281
Yao, Y., Yang E., Pan Y., Shu X., Liu G. Mining an O-methyltransferase for de novo biosynthesis of physcion in Aspergillus nidulans, Applied Microbiology and Biotechnology, 2023, 107 (4): 1177-1188.

1.-31. (canceled)

32. An O-methyltransferase enzyme, wherein said O-methyltransferase enzyme has O-methyltransferase activity on an anthraquinone and comprises an amino acid sequence having at least 60% identity to an amino acid sequence as defined in any of SEQ ID NO:1, SEQ ID NO:2, or SEQ ID NO:3.

33. The O-methyltransferase enzyme according to claim 32, wherein the amino acid sequence has at least 70% identity to an amino acid sequence as defined in any of SEQ ID NO:1, SEQ ID NO:2, or SEQ ID NO:3.

34. The O-methyltransferase enzyme according to claim 32, wherein the amino acid sequence has at least 80% or at least 90% identity to an amino acid sequence as defined in any of SEQ ID NO:1, SEQ ID NO:2, or SEQ ID NO:3.

35. The O-methyltransferase enzyme according to claim 32, wherein said O-methyltransferase enzyme comprises or consists of an amino acid sequence as defined in any of SEQ ID NO:1, SEQ ID NO:2, or SEQ ID NO:3.

36. The O-methyltransferase enzyme according to claim 32, wherein the anthraquinone is selected from the group consisting of endocrocin and endocrocin analogues.

37. The O-methyltransferase enzyme according to claim 32, wherein the anthraquinone is selected from the group consisting of emodin and emodin analogues.

38. An isolated nucleic acid molecule, wherein the isolated nucleic acid molecule comprises a polynucleotide sequence which encodes an O-methyltransferase enzyme and is selected from the group consisting of:
a) a nucleic acid molecule encoding a polypeptide having O-methyltransferase activity on an anthraquinone and comprising the amino acid sequence as defined in SEQ ID NO: 1,
b) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 1,
c) a nucleic acid molecule encoding a polypeptide having O-methyltransferase activity on an anthraquinone and comprising the amino acid sequence as defined in SEQ ID NO: 2,
d) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 2,
e) a nucleic acid molecule encoding a polypeptide having O-methyltransferase activity on an anthraquinone and comprising the amino acid sequence as defined in SEQ ID NO: 3,
f) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 3.

39. The isolated nucleic acid molecule according to claim 38, wherein the anthraquinone is selected from the group consisting of endocrocin, endocrocin analogues, emodin, and emodin analogues.

40. The recombinant expression vector, wherein the expression vector comprises the isolated nucleic acid according to claim 38, operably linked to at least one regulatory sequence capable of directing expression of said O-methyltransferase enzyme in a suitable host.

41. A host cell, wherein the host cell comprises one of:
a nucleic acid molecule comprising a polynucleotide sequence which encodes an O-methyltransferase enzyme and is selected from the group consisting of:
a) a nucleic acid molecule encoding a polypeptide having O-methyltransferase activity on an anthraquinone and comprising the amino acid sequence as defined in SEQ ID NO: 1,
b) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 1,
c) a nucleic acid molecule encoding a polypeptide having O-methyltransferase activity on an anthraquinone and comprising the amino acid sequence as defined in SEQ ID NO: 2,
d) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 2,
e) a nucleic acid molecule encoding a polypeptide having O-methyltransferase activity on an anthraquinone and comprising the amino acid sequence as defined in SEQ ID NO: 3,
f) a nucleic acid molecule encoding a polypeptide having O-methyltransferase activity on an anthraquinone and at least 60% identity, preferably at least 70% identity, more preferably at least 80% identity, most preferably at least 90% identity to the amino acid sequence as defined in SEQ ID NO: 3,
or a recombinant expression vector comprising the isolated nucleic acid operably linked to at least one regulatory sequence capable of directing expression of said O-methyltransferase enzyme in a suitable host.

42. The host cell according to claim 41, wherein the host cell is selected from the group consisting of a bacterial cell, a yeast cell, a filamentous fungus cell, a plant cell, and a non-human animal cell, preferably the host cell is a filamentous fungus cell or a yeast cell.

43. The host cell according to claim 41, wherein the host is selected from the group consisting of a genus Saccharomyces, Aspergillus, Trichoderma, and Yarrowia.

44. The host cell according to claim 41, wherein the host cell is selected from a group of cells that can produce an anthraquinone.

45. The host cell according to claim 41, wherein the host cell is selected from a group of cells that can produce endocrocin, an endocrocin analogue, emodin, or an emodin analogue.

46. The host cell according to claim 41, wherein the host cell is selected from the group consisting of Saccharomyces cerevisiae, Aspergillus oryzae, Trichoderma reesei, and Yarrowia lipolytica.

47. An enzyme preparation, wherein the enzyme preparation comprises O-methyltransferase enzyme according to claim 32.

48. A process of producing a compound which is an O-methylanthraquinone, wherein the process comprises the steps of:
providing an anthraquinone;
providing a recombinant host cell according to claim 41;
culturing the host cell under conditions to produce an O-methyltransferase which converts the anthraquinone to an O-methylanthraquinone, wherein the host cell comprises a nucleic acid sequence encoding O-methyltransferase exhibiting activity for catalyzing an O-methylation reaction on said anthraquinone;
converting enzymatically the anthraquinone to the O-methylanthraquinone; and
optionally recovering said O-methylanthraquinone.

49. The process according to claim 48, wherein the O-methylanthraquinone is selected from the group consisting of dermolutein, physcion, dermolutein analogues, and physcion analogues.

50. The process according to claim 48, wherein the anthraquinone is selected from the group consisting of endocrocin and endocrocin analogues.

51. The process according to claim 48, wherein the anthraquinone is selected from the group consisting of emodin and emodin analogues.

52. The process of claim 48 further comprising the step of using the O-methylanthraquinone as a colorant in textiles, plastics, pigments, dyes, food, feed, or cosmetics industries, or any related industries.

53. A colorant composition, wherein said composition comprises the O-methylanthraquinone obtained according to the process of claim 48.
